(19) United States
(12) Patent Application Publication
(10) Pub. No.: US 2023/0002484 A1
(43) Pub. Date: Jan. 5, 2023

(54) TREATMENT OF ATOPIC DERMATITIS EMPLOYING ANTI-IL-13Ra1 ANTIBODY OR BINDING FRAGMENT THEREOF

(71) Applicant: Aslan Pharmaceuticals PTE LTD, Singapore (SG)

(72) Inventors: Karen VEVERKA, Singapore (SG); Alison WARD, Singapore (SG)

(21) Appl. No.: 17/929,824

(22) Filed: Sep. 6, 2022

Related U.S. Application Data
(63) Continuation-in-part of application No. PCT/SG2022/050102, filed on Mar. 1, 2022.

(30) Foreign Application Priority Data
Mar. 1, 2021 (SG) 10202102086S
Sep. 27, 2021 (SG) 10202110688T

(51) Int. Cl. C07K 16/24

(52) U.S. Cl. CPC C07K 16/244; A61K 2039/545

(57) ABSTRACT

An antibody, antigen binding fragment thereof or a pharmaceutical formulation, which is an inhibitor of signalling through IL-13Rα1 by binding said receptor and compositions comprising the same for use in the treatment of atopic dermatitis (for example moderate to severe atopic dermatitis, in particular poorly controlled moderate to severe atopic dermatitis) by parenteral administration of a treatment cycle comprising a dose in the range 200 mg to 600 mg, (such as 400 to 600 mg), wherein the disease is modified by a percentage reduction in EASI score in the range −20 to −100% from the baseline.

### CROSS-REFERENCE TO RELATED APPLICATIONS

This application is a Continuation-in-Part of International Patent Application No. PCT/SG2022/050102 filed on Mar. 1, 2022, which claims priority to Singapore Application No. SG10202110688T filed on Sep. 27, 2021 and Singapore Application No. SG10202102086S filed on Mar. 1, 2021, the content of each of which applications is incorporated herein by reference.

### INCORPORATION OF SEQUENCE LISTING

This application contains a sequence listing submitted electronically via EFS-web, which serves as both the paper copy and the computer readable form (CRF) and consists of a file entitled “STAPL16CIP_seqlistxml”, which was created on Sep. 6, 2022, which is 83,245 bytes in size, and which is herein incorporated by reference in its entirety.

The present disclosure relates to use of an anti-IL-13Rα1 antibody or a binding fragment thereof and pharmaceutical formulations comprising same to treat patients with atopic dermatitis to stimulate disease modification.

### BACKGROUND

One way to inhibit the activity of IL-13 is to interfere with the binding of IL-13 to its receptor IL-13R, for example by using an antibody specific to IL-13R, such as an antibody specific to IL-13Rα1. An effective antibody antagonist to IL-13Rα1 may also interfere with the binding of IL-13 and prevent heterodimerization of IL-4Rα and IL-13Rα1. Such an antibody could inhibit signaling of both IL-13 and IL-4 through the type II receptor while sparing IL-4 signalling through the type I receptor. Signalling through the type I receptor is essential in the induction phase of the immune response during which Th2 cells differentiate. T cells do not express IL-13Rα1 so the type II receptor plays no role in Th2 differentiation. Hence, an IL-13Rα1 antibody should not affect the overall Th1/Th2 balance. Signalling through the type II IL-4/IL-13 receptor is critical during the effector-A-stage of the immune response during established allergic inflammation. Thus, blockade of the type II receptor should have a beneficial effect on many of the symptoms of conditions mediated by IL-13R-mediated and therefore, be an effective disease modifying agent.

Antibodies against IL-13Rα1 (both monoclonal and polyclonal) have been described in the art; see, eg, WO 97/15663, WO 03/80675; WO 03/46009; WO 06/072564; Gauchat et al, 1998 Eur. J. Immunol. 28: 4286-4298; Gauchat et al, 2000 Eur. J. Immunol. 30: 3157-3164; Clement et al, 1997 Cytokine 9(11): 959 (Meeting Abstract); Ogata et al, 1998 J. Biol. Chem. 273: 9864-9871; Graber et al, 1998 Eur. J. Immunol. 28: 4286-4298; C. Vermot-Desroches et al, 2000 Tissue Antigens 5(Supp. 1): 52-53 (Meeting Abstract); Poudrier et al, 2000 Eur. J. Immunol. 30: 3157-3164; Akaiwa et al, 2001 Cytokine 13: 75-84; Cancino-Diaz et al, 2002 J. Invest Dermatol. 119: 1114-1120; and Krause et al, 2006 MoI. Immunol. 43: 1799-1807.

One particularly promising anti-IL-13Rα1 antibody is described in WO2008/060813 as antibody 10G5-6. 10G5-6 as an IgG4 with a hinge stabilising serine to proline mutation (S241P Kabat numbering) is known as ASLAN004. ASLAN004 has been shown to bind to human IL-13Rα1 with a high affinity (for example Kd may be 500 pM). ASLAN004 was shown to effectively antagonise IL-13 function through inhibiting the binding of IL-13 to its receptor IL-13Rα1 and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells. Atopic dermatitis can be a very painful, demoralising and psychologically damaging disease. One method of assessing the disease is the EASI score. The score is in the range 0-72.

In some instances, moderate to severe forms of the disease are not adequately controlled by topical medicines. In addition, it is not advisable for some patients to take the available topical medicines. Dupixent (dupilumab) is an antibody inhibitor of the interleukin-4 receptor alpha (IL-4Rα), which is licensed for the treatment of atopic dermatitis.

At phase 2b, at a dose of 300 mg of dupilumab once a week, at day 57, fifty percent of patients had EASI 75 (a 75% reductions from baseline) and thirty percent of patients had an EASI 90 (a 90% percent reduction from baseline).

In contrast it may be that higher levels of disease modification can be achieved in the same or shorter timeframes by targeting IL-13Rα1.

### SUMMARY OF THE DISCLOSURE

1. An antibody, antigen binding fragment thereof or a pharmaceutical formulation comprising same, which is an inhibitor of signalling through IL-13Rα1 by binding the said receptor, for use in the treatment of atopic dermatitis (for example moderate to severe atopic dermatitis, in particular poorly controlled moderate to severe atopic dermatitis) by parenteral administration of a treatment cycle comprising a dose in the range 200 mg to 600 mg, (such as 400 to 600 mg), wherein the disease is modified by a percentage reduction in EASI score in the range −20 to −100% from the baseline.
1A. An antibody, antigen binding fragment thereof or a pharmaceutical formulation, which is an inhibitor of signalling through IL-13Rα1 by binding the said receptor, for use in the treatment to reduce EASI score in the range −20 to −100% from the baseline in a patient with atopic dermatitis, for example moderate to severe atopic dermatitis (in particular poorly controlled moderate to severe atopic dermatitis) by parenteral administration of a treatment cycle comprising a dose in the range 200 mg to 600 mg, (such as 400 to 600 mg).
2. An antibody, antigen binding fragment thereof or a pharmaceutical formulation according to paragraph 1 or 1A, wherein reduction in EASI score is present after about two weeks from administration of the first dose (such as day 15).
3. An antibody, antigen binding fragment thereof or a pharmaceutical formulation according to paragraph 1, 1A or 2, wherein reduction in EASI score is present after about four weeks from administration of the first dose (such as day 29).
4. An antibody, antigen binding fragment thereof or a pharmaceutical formulation according to any one of paragraphs 1, 1A to 3, wherein reduction in EASI score is present after about six weeks from administration of the first dose (such as day 43).
5. An antibody, antigen binding fragment thereof or a pharmaceutical formulation according to any one of paragraphs 1, 1A to 4, wherein reduction in EASI score is present after about eight weeks from administration of the first dose (such as day 57).
6. An antibody, antigen binding fragment thereof or a pharmaceutical formulation according to any one of paragraphs 1, 1A to 5, wherein the treatment is administered intravenously.
7. An antibody, antigen binding fragment thereof or a pharmaceutical formulation according to any one of paragraphs 1, 1A to 5, wherein the treatment is administered subcutaneously.
8. An antibody, antigen binding fragment thereof or a pharmaceutical formulation according to any one of paragraphs 1, 1A to 7, wherein multiple doses are administered in a treatment cycle (for example wherein the treatment cycle is 4 to 8 weeks, such as 8 weeks).
9. An antibody, binding fragment thereof or a pharmaceutical formulation according to paragraph 8, wherein multiple treatment cycles are administered, for example 2, 3, 4 or more treatment cycles are administered.
10. An antibody, binding fragment thereof or a pharmaceutical formulation according to paragraph 8 or 9, wherein following the treatment cycle or cycles and disease modification, maintenance therapy is administered, for example the same dose administered less frequently (for example monthly), or a lower dose (such as 200 mg) administered the same frequency or less frequently (such as about two weekly, about three weekly, or about four weekly.
11. An antibody, antigen binding fragment or a pharmaceutical formulation according to any one of paragraphs 1, 1A to 7, wherein said antibody or binding fragment thereof is administered approximately weekly, (in particular a single treatment cycle, especially 8 weeks).
12. An antibody, antigen binding fragment or a pharmaceutical formulation according to any one of paragraphs 1, 1A to 10, wherein said antibody or binding fragment thereof is administered once approximately every two weeks, (in particular a single treatment cycle, especially 8 weeks).
13. An antibody, antigen binding fragment or a pharmaceutical formulation according to any one of paragraphs 1, 1A to 10, wherein said antibody or binding fragment thereof is administered once approximately every three weeks, (in particular a single treatment cycle, especially 8 weeks).
14. An antibody, antigen binding fragment or a pharmaceutical formulation according to any one of paragraphs 1, 1A to 10, wherein the antibody or binding fragment thereof is administered once approximately every four weeks (for example monthly), (in particular a single treatment cycle, especially 8 weeks).
15. An antibody, antigen binding fragment or a pharmaceutical formulation according to any one of paragraphs 1, 1A to 14, wherein a loading dose in the range 400 to 900 mg, for example 400, 500, 600, 700, 800 or 900 mg is employed before administration of the treatment cycle.
16. An antibody, antigen binding fragment or a pharmaceutical formulation according to any one of paragraphs 1, 1A to 14, wherein the treatment does not comprise a loading dose.
17. An antibody or antigen binding fragment thereof according to any one of paragraphs 1 to 16, wherein the dose is 200 mg.
18. An antibody, antigen binding fragment thereof or a pharmaceutical formulation according to paragraph 17, wherein the reduction in EASI is in the range −15 to −60% (for example at about day 15).
19. An antibody, antigen binding fragment thereof or a pharmaceutical formulation according to paragraph 17 or 18 wherein the reduction in EASI score is in the range −40 to −85% (for example at about day 29).
20. An antibody, antigen binding fragment thereof or a pharmaceutical formulation according to any one of paragraphs 17 to 19, wherein the reduction in EASI score is in the range −25 to −85% (for example at about day 43 or 57).
21. An antibody, antigen binding fragment thereof or a pharmaceutical formulation according to any one of paragraphs 1, 1A to 16, wherein the dose is in the range 350 to 450 mg, such as 400 mg, for example wherein 80% of the patient population has an EASI 50 at about day 29 and/or day 57.
22. An antibody, antigen binding fragment thereof or a pharmaceutical formulation according to any one of paragraphs 1 to 16, wherein the dose is 600 mg.
23. An antibody, antigen binding fragment thereof or a pharmaceutical formulation according to paragraph 21 or 22, wherein the reduction in EASI score is in the range −25 to −60% (for example −39 to −59, such as −40 to −59, in particular −47, −48, −49, −50, −51, −52, −53, −54, −55, −56, −57, −58 or −59%) for example at about day 15.
24. An antibody, antigen binding fragment thereof or a pharmaceutical formulation according to any one of paragraphs 21 to 23, wherein the reduction in EASI score is in the range −50 to −100% (for example −55 to −97%) in particular at about day 29.
25. An antibody, antigen binding fragment thereof or a pharmaceutical formulation according to any one of paragraphs 21 to 24, wherein the reduction in EASI score is in the range −60 to −100% (for example −70 to −97%) in particular at about day 43.
26. An antibody, antigen binding fragment thereof or a pharmaceutical formulation according to any one of paragraphs 21 to 25, wherein the reduction in EASI score is in the range −65 to −100% (for example −70 to −100, such as −90 to −-100, in particular 91, 92, 93, 94, 95, 96, 97, 98, 99 or 100%) in particular at about day 57.
27. An antibody, binding fragment thereof or a pharmaceutical formulation according to any one of paragraphs 21 to 26, wherein 90% of the patient population has an EASI 50 at about day 57.
28. An antibody, antigen binding fragment thereof or a pharmaceutical formulation according to any one of paragraphs 1, 1A to 26, wherein the treatment cycles comprises, a first dose at 600 mg, followed by three weekly doses of 400 mg, for example wherein the treatment cycle is repeated twice i.e. two treatment cycles lasting 8 weeks, in particular day 1 600 mg, approximately day 8 400 mg, approximately day 15 400 mg, approximately day 22 400 mg, approximately day 29 600 mg, approximately day 36 400 mg, approximately day 43 400 mg, and approximately day 50 400 mg are administered.
29. An antibody, antigen binding fragment thereof or a pharmaceutical formulation according to any one of paragraphs 1, 1A to 28, wherein disease modification, occurs by day 4, wherein day 1 is the first administration of the antibody or binding fragment thereof.
30. An antibody, antigen binding fragment or a pharmaceutical formulation according to paragraph 29, wherein the disease modification is a reduction in EASI score, for example wherein the reduction is a percentage from base line in the range −10 to 55%.
31. An antibody, antigen binding fragment thereof or a pharmaceutical formulation according to any one of paragraphs 1, 1A to 30, wherein the disease modification in the range −40 to -100% is achieved by about day 57 following first administration on day 1, for example maximum disease modification is achieved by about day 57.
32. An antibody, antigen binding fragment thereof or a pharmaceutical formulation for use according to any one of paragraphs 1, 1A to 30, wherein the antibody or binding fragment binds an epitope FFYQ (for example same epitope as the antibody with a VH shown in SEQ ID NO: 51 and a VL shown in SEQ ID NO: 53, or a sequence at least 95% identical to any one of the same.
33. An antibody, antigen binding fragment thereof or a pharmaceutical formulation for use according to any one of paragraphs 1, 1A to 32, wherein the anti-IL-13R antibody comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10.
34. An antibody, antigen binding fragment thereof or a pharmaceutical formulation for use according to any one of paragraphs 1, 1A to 33, wherein the anti-IL-13R antibody comprises a VH domain comprising an amino acid sequence shown in SEQ ID NO: 51 or a sequence at least 95% identical thereto, in particular SEQ ID NO: 51.
35. An antibody or antigen binding fragment thereof for use according to any one of paragraphs 1, 1A to 34, wherein the anti-IL-13R antibody comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
36. An antibody or antigen binding fragment thereof for use according to any one of paragraphs 1, 1A to 35, wherein the anti-IL-13R antibody comprises a VL domain comprising an amino acid sequence shown in SEQ ID NO: 53 or a sequence at least 95% identical thereto, in particular SEQ ID NO: 53.
37. An antibody or antigen binding fragment thereof for use according to any one of paragraphs 1, 1A to 36, wherein the antibody is provided as a pharmaceutical formulation comprising

10 to 140 mg/ml of the antibody or binding fragment;

50 mM to 150 mM of arginine (for example 50, 55, 60, 65, 70, 75, 80, 85, 90, 95, 100, 105, 110, 115, 120, 125, 130, 135, 140, 145 or 150, such as 100 mM arginine);

15 to 25 mM histidine buffer, for example 15, 16, 17, 18, 19, 20, 21, 22, 23, 24 and 25, such as 20 mM histidine buffer;

0.01-0.03% of a non-ionic surfactant, such as 0.02% w/v and

wherein the pH of the formulation is in the range 5.5 to 7.5 for example 6.2 to 7.2 (such as 6.2, 6.3, 6.4, 6.5, 6.6, 6.7, 6.8, 6.9, 7.0, 7.1, 7.2), such as 6.5 to 7.0, in particular 6.4 to 6.9)

38. An antibody, antigen binding fragment thereof or a pharmaceutical formulation for use according to paragraph 37, wherein the osmolarity of the formulation is in the range 350 to 550 mOsmo/kg, for example 350, 355, 365, 370, 375, 380, 385, 390, 395, 400, 405, 410, 415, 420, 425, 430, 435, 440, 445, 450, 455, 460, 465, 470, 475, 480, 485, 490, 495, 500, 505, 515, 520, 525, 530, 535, 540, 545, 550, such as 405 to 435 mOsmo/kg.
39. An antibody, antigen binding fragment thereof or a pharmaceutical formulation for use according to paragraphs 37 or 38, which further comprises 50 to 200 mM of a sugar, for example 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, such as 180 mM sugar.
40. An antibody, antigen binding fragment or a pharmaceutical formulation for use according to any one of paragraphs 37 to 40, wherein the pH is 6.5.
41. An antibody, antigen binding fragment thereof or a pharmaceutical formulation for use according to any one of paragraphs 37 to 40, wherein the formulation does not comprise NaCl.
42. An antibody, antigen binding fragment thereof or a pharmaceutical formulation for use according to any one of paragraphs 37 to 41, wherein the formulation comprises 50 to 150 mM of NaCl, for example50, 55, 60, 65, 70, 75, 80, 85, 90, 95, 100, 105, 110, 115, 120, 125, 130, 135, 140, 145, 150, such as 62.5 or 140 mM NaCl.
43. A method of treating a patient for atopic dermatitis (for example moderate to severe atopic dermatitis, in particular poorly controlled moderate to severe atopic dermatitis) comprising administering parenterally an antibody, antigen binding fragment thereof or a pharmaceutical formulation, which is an inhibitor of signalling through of the IL-13Rα1 by binding the said receptor (for example according to any one of paragraphs 1 to 42), such that the incidence of side effects in the eyes are reduced in comparison to treatment with the therapeutic dose of dupilumab for treatment of the same.
44. Use of an antibody, antigen binding fragment or a pharmaceutical formulation, which is an inhibitor of signalling through of the IL-13Rα1 by binding the said receptor, for example according to any one of paragraphs 1 to 42, for use in the manufacture of a medicament for the treatment of atopic dermatitis wherein the incidence of side effects in the eyes are reduced in comparison to treatment with the therapeutic dose of dupilumab for treatment of the same

Also provided is method of treating a patient for atopic dermatitis (for example moderate to severe atopic dermatitis, in particular poorly controlled moderate to severe atopic dermatitis) according to the present disclosure comprising administering an antibody or antigen binding fragment thereof, or pharmaceutical formulation disclosed herein.

In further aspect there is provided use of an antibody or antigen binding fragment or a pharmaceutical formulation disclosed herein for use in the manufacture of a medicament for the treatment of atopic dermatitis according to the present disclosure.

In one embodiment there is a provided a reduction in the Investigator Global Assessment IGA with/after treatment according to the present disclosure, for example an assessment of 0, 1 or 2, (no inflammatory signs, almost clear and mild disease respectively). In particular there is provided an IGA score of 0 or 1.

In one embodiment there is provided a reduction in percentage body surface area (%BSA) of atopic dermatitis (AD) involvement with/after treatment according to the present disclosure, for example a reduction of at least −20% from the baseline (i.e. at least −20% CFBL), such as a −20%, −30%, −40%, −50%, −60%, −70%, −80%, −90% or −100% CFBL. In one embodiment, there is a reduction in percentage body surface area (% BSA) of AD involvement of −40 to −70%, such as −40%, −50%, −60% or −70%. In one embodiment, there is a reduction in % BSA of at least −50%, such as −50%, −55%, −60% or −65%.

In one embodiment combination therapy is employed comprising the antibody, antigen binding fragment thereof or a formulation according to the present disclosure and a further medicament. In one embodiment the further medicament is for the treatment of atopic dermatitis, for example topical steroids, oral steroids, and/or antihistamines.

Surprisingly disease modification following treatment with an anti IL-13Rα1 antibody or binding fragment thereof according to the present disclosure closely follows reduction in TARC, in fact the TARC reduction and EASI reduction seem to correlate closely.

### DETAILED DISCLOSURE

Disease modification as employed herein relates to improvements in the disease status, for example as measured by any suitable clinical parameter, in particular a reduction in the EASI score.

A clinically relevant score is a score used in the clinical, for example used by a physician.

In one embodiment the disease is modified by a percentage reduction in Eczema Area and Severity Index (EASI) score in the range −20 to −100% from the baseline, such as EASI 50, EASI 75 or EASI 90.

EASI score and EASI are used interchangeably herein.

Eczema Area and Severity Index (EASI) score as used herein is a tool used to measure the area (which indicates the extent of disease) and severity of atopic eczema. The number after the term “EASI” indicates the % decrease in the score from baseline. Thus, EASI 50 for example refers to 50% decrease in the score and EASI 90 refers to a 90% decrease in the score.

In one embodiment disease modification is measured as a reduction in IGA.

Investigator's global assessment (IGA) as used herein refers to a tool for the assessment of atopic dermatitis. It uses a 0-5 point scale depending on the severity of a patient's symptoms:

| Score | Severity | Description of symptoms |
|---|---|---|
| 0 | clear | no inflammatory signs |
| 1 | almost clear | Just perceptible erythema and just perceptible |
| | | papulation/infiltration |
| 2 | mild disease | Mild erythema and mild papulation/infiltration |
| 3 | moderate disease | moderate erythema and moderate papulation/infiltration |
| 4 | severe disease | severe erythema and severe papulation/infiltration |
| 5 | very severe disease | severe erythema and severe papulation/infiltration with |
| | | oozing/crusting |

Body surface area (BSA) of atopic dermatitis (AD) involvement as used herein refers to a simple measure of percentage body surface area (%BSA) involved with atopic dermatitis, which does not incorporate disease severity.

Patient Oriented Eczema Measure (POEM) as used herein refers to a 7-item questionnaire for patients that assesses presence of disease symptoms (dryness, itching, flaking, cracking, sleep loss, bleeding, and weeping) over the last week using a scoring system of 0 (no days) to 4 (every day). The total score ranges from 0 to 28 with higher scores indicating greater intensity of eczema.

Interleukin-13 receptor (IL-13R) as used herein is a type I cytokine receptor, which binds to Interleukin-13. It consists of two subunits, encoded by IL13Rα1 and IL4R, respectively. These two genes encode the proteins IL-13Rα1 and IL-4Rα. These form a dimer with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STATE. Human IL-13Rα1 has the Uniprot number P3597.

IL-13Rα2, previously called IL-13R and IL-13Rα, is another receptor which is able to bind to

IL-13. However, in contrast to IL-13Rα1, this protein binds IL-13 with high affinity, but it does not bind IL-4. Human IL-13Rα2 has the Uniprot number Q14627.

In one embodiment the anti-IL-13R antibody or binding fragment thereof of the present disclosure binds to IL-13Rα1. In one embodiment, the antibody or binding fragment thereof binds only to IL-13Rα1 and does not bind to IL-13Rα2.

In one embodiment CDRH1 comprises an amino acid sequence GYSFTSYWIG (SEQ ID NO: 1) as disclosed in WO2020/197502 incorporated herein by reference.

In one embodiment CDRH2 comprises an amino acid sequence VIYPGDSYTR (SEQ ID NO: 2) as disclosed in WO2020/197502 incorporated herein by reference.

In one embodiment CDRH3 comprises the formula:

SEQ ID NO: 3
X₁ Pro Asn Trp Gly X₆ X₇ Asp X₉

X₁ denotes Phe, Met, Gln, Leu or Val
X₆ denotes Ser or Ala
X₇ denotes Phe, Leu, Ala or Met
X₉ denotes Tyr, Gln, Lys, Arg Trp, His, Ala, Thr, Ser, Asn or Gly
   In one embodiment the IL13-R1α1 antibody or binding fragment employed in the formulation of the present disclosure comprises a CDRH3 independently selected from a sequence comprising SEQ ID NO: 4 to 30.

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: or 3.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

In one embodiment CDRL1 is an amino acid sequence comprising RASQSISSSYLA SEQ ID NO: 31.

In one embodiment CDRL2 is an amino acid sequence comprising GASSRAT SEQ ID NO: 32

In one embodiment CDL3 comprises the formula:

SEQ ID NO: 33
Gln X₂X₃X₄X₅

X₂ denotes Gln, Arg, Met, Ser, Thr or Val.
X₃ denotes Tyr or Val.
X₄ denotes Glu, Ala, Gly or Ser.
X₅ denotes Thr, Ala or Ser.
   In one embodiment the IL-13Rα1 antibody employed in the formulation of the present disclosure comprises a CDRL3 independently selected from a sequence comprising SEQ ID NO: 34 to 47:

In one embodiment, the anti-IL-13Rα antibody or binding fragment employed in the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33.

In one embodiment, the anti-IL-13Rα antibody of the present disclosure comprises a VL CDR1 comprising an amino acid sequence SEQ ID NO: 31, a VL CDR2 comprising an amino acid sequence SEQ ID NO: 32, and a VL CDR3 comprising an amino acid sequence as set forth in SEQ ID NO: 34 35, 36, 37, 38, 39, 40, 41, 42, 43, 44, 45, 46, or 47.

In one embodiment, the anti-IL-13Rα antibody of the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: or 3, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 34, 35, 36, 37, 38, 39, 40, 41, 42, 43, 44, 45, 46, or 47.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment the VH region is independently selected from the group comprising: SEQ

ID NO: 48, SEQ ID NO: 49, SEQ ID NO: 50 and SEQ ID NO: 51, or a sequence at least 95% identical to any one of the same, in particular any sequence independently selected from SEQ ID NO: 48, 49, 50 and 51.

SEQ ID NO: 48
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
FPNWGSFDYWGQGTLVTVSS
SEQ ID NO: 49
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSFDYWGQGTLVTVSS
SEQ ID NO: 50
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCVR
MPNWGSLDHWGQGTLVTVSS
SEQ ID NO: 51
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSS

In one embodiment the VL is independently selected from the group comprising SEQ ID NO: 52, SEQ ID NO: 53 and SEQ ID NO: 54, or a sequence at least 95% identical to any one of the same, in particular a sequence independently selected from SEQ ID NO: 52, 53 and 54.

SEQ ID NO: 52
EIVLTQSPGTLSLSPGERATLSCRASQSISSSYLAWYQQKPGQAPRLLIY
GASSRATGIPDRFSGSGSGTDFTLTISRLEPEDFAVYYCQQYETFGQGTK
VEI*
SEQ ID NO: 53
EIVLTQSPGTLSLSPGERATLSCRASQSISSSYLAWYQQKPGQAPRLLIY
GASSRATGIPDRFSGSGSGTDFTLTISRLEPEDFAVYYCQQYASFGQGTK
VEI*
SEQ ID NO: 54
EIVLTQSPGTLSLSPGERATLSCRASQSISSSYLAWYQQKPGQAPRLLIY
GASSRATGIPDRFSGSGSGTDFTLTISRLEPEDFAVYYCQQYEAFGQGTK
VEI*
(*K deleted in a post translational modification).

SEQ ID NO: 1 to 54 are disclosed in WO2020/197502 incorporated herein by reference.

In one embodiment the VH sequence is SEQ ID NO: 48 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VH sequence is SEQ ID NO: 49 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VH sequence is SEQ ID NO: 50 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VH sequence is SEQ ID NO: 51 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51. (or a sequence at least 95% identical to any one of the same)

In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VH sequence is SEQ ID NO: 51 (or a sequence at least 95% identical thereto) and the VL sequence is SEQ ID NO: 53 ((or a sequence at least 95% identical thereto).

Variable region as employed herein refers to the region in an antibody chain comprising the CDRs and a suitable framework.

In one embodiment the heavy chain comprises a sequence independently selected from the group comprising SEQ ID NO: 55, SEQ ID NO: 56, SEQ ID NO: 57, SEQ ID NO: 58, SEQ ID NO: 59 and SEQ ID NO: 60, or a sequence at least 95% identical to any one of the same.

Each SEQ ID NO: 55 to 60 have a post translational modification, which is deletion of K at the C terminal. SEQ ID NO: 55 to 60 are disclosed as SEQ ID NO: 56 to 61 in W02020/197502, incorporated herein by reference.

SEQ ID NO: 61, 62 and 63 herein are disclosed in WO2020/197502, incorporated herein by reference, as SEQ ID NO: 62, 63 and 55.

In one embodiment the heavy chain is independently selected from SEQ ID NO: 55, 56, 57, 58, 59, and 60 (or a sequence at least 95% identical to any one of the same) and the light chain is independently selected from SEQ ID NO: 61, 62 and 63 (or a sequence at least 95% identical to any one of the same).

In one embodiment the heavy chain is SEQ ID NO: 55 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 56 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 57 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 58 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 59 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 60 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 58 or 60 (or a sequence at least 95% identical to any one of the same) and a light chain with the sequence shown in SEQ ID NO: 61 (or a sequence at least 95% identical thereto).

In one embodiment the heavy chain is SEQ ID NO: 58 (or a sequence at least 95% identical to any one of the same) and a light chain with the sequence shown in SEQ ID NO: 61 (or a sequence at least 95% identical thereto).

In one embodiment the heavy chain is SEQ ID NO: 60 (or a sequence at least 95% identical to any one of the same) and a light chain with the sequence shown in SEQ ID NO: 61 (or a sequence at least 95% identical thereto).

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Any discussion of a protein, antibody or amino acid sequence herein will be understood to include any variants of the protein, antibody or amino acid sequence produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” (C-terminal lysine residues of encoded antibodies are often removed during the manufacturing process) and/or have part or all of a signal sequence incompletely processed and, as a consequence, remain at the terminus of the antibody. It is understood that an antibody comprising a particular amino acid sequence or binding fragment thereof may be a heterogeneous mixture of the stated or encoded sequence and/or variants of that stated or encoded sequence or binding fragment thereof.

In one embodiment the present disclosure extends to a sequence explicitly disclosed herein where the C-terminal lysine (K) has been cleaved.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see eg U.S. Pat. No. 5,585,089 & WO91/09967). It will be appreciated that it may only be necessary to transfer the specificity determining residues of the CDRs rather than the entire CDR (see eg, Kashmiri et al., 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al.,). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human.

Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP0438474 and EP0463151.

Thus, the presently disclosed anti-IL13R antibody may comprise one or more constant regions, such as a naturally occurring constant domain or a derivate of a naturally occurring domain.

A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or indirectly including via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62: 119-58 and Dubowchik et al , 1999, Pharmacology and Therapeutics, 83, 67-123). Particular chemical procedures include, for example, those described in WO93/06231, WO92/22583, WO89/00195, WO89/01476 and WO03/031581. Alternatively, where the effector molecule is a protein or polypeptide the linkage may be achieved using recombinant DNA procedures, for example as described in WO86/01533 and EP0392745.

The term effector molecule as used herein includes, for example, biologically active proteins, for example enzymes, other antibody or antibody fragments, synthetic or naturally occurring polymers, nucleic acids and fragments thereof eg DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero- polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 55,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In patients with cancer, such as breast cancer, cancer related lymphedema (BCRL), the formulation of the present disclosure may prevent lymphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lympheclema after lymphatic injury is associated with tissue inflammation, the infiltration of CD4-positive cells and their differentiation to the type 2 helper T-cell (Th2) phenotype, Th2 cells produce IL-4 and IL-13 that play a key role in the development of lymphedema-associated symptoms as well as other Th2-mediated diseases.

In one embodiment the formulation herein is administered in combination with another therapy.

“In combination” as employed herein is intended to encompass where the anti-IL13R antibody is administered before, concurrently with another therapy or after another therapy, as the same or different formulations. Thus, combination is where the pharmacological effect of a first therapy exists at the same as the existence of a pharmacological effect of second therapy in the body and/or the two therapies are part of treatment plan designed to be employed together.

Therapeutic dose as employed herein refers to the amount of the anti-IL13R antibody, such as ASLAN004, that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment a formulation according to the present disclosure (including a formulation comprising same) is administered monthly, for example in a treatment cycle or as maintenance therapy.

Unit dose as used herein generally refers to a product comprising the amount of anti-IL13R antibody or binding fragment thereof of the present disclosure that is administered in a single dose including any overage.

A unit dose of the presently claimed anti-IL13R antibody or antigen binding fragment thereof may refer to the marketed form of the product, such as a formulation of the anti-IL13R antibody or binding fragment thereof, wherein the product is apportioned into the amount of anti-IL13R antibody that is required for a single dose. Thus, the manufacturer is able to determine and control the exact amount of anti-13R antibody or binding fragment thereof to be included in each unit dose.

The product may be in various forms, familiar to the skilled addressee, such as vials, ampoules, infusion bags or a device (including an auto-injection device).

The exact amount as employed herein refers to the amount to be administer as a dose to the patient and any overage.

In one embodiment, the unit dose or unit doses are for use according to a method of the present disclosure.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section of this specification contains relevant technical information and may be used as basis for amendment. Subject headings herein are employed to divide the document into sections and are not intended to be used to construe the meaning of the disclosure provided herein.

The present specification claims priority from SG10202102086S (filed 01 Mar. 2021); SG10202110688T (filed 27 Sep. 2021) both incorporated herein by reference. These applications may be used as basis for corrections to the present specification, especially in respect of sequences disclosed therein.

The present invention is further described by way of illustration only in the following examples.

### BRIEF DESCRIPTION OF FIGURES

FIG. 1A Table showing demographics of full analysis set

FIG. 1B Table showing baseline disease characteristics of full analysis set

FIG. 1C Table showing baseline disease characteristics of Evaluable for Efficacy set (EES)

FIG. 2A Table showing % change from baseline in EASI score at Day 57 for EES

FIG. 2B Graph showing % change from baseline in EASI score at Day 57 for EES (ASLAN004 200 mg, 400 mg and 600 mg)

FIG. 2C Graph showing % change from baseline in EASI score at Day 57 for EES (ASLAN004 low dose and high doses)

FIG. 3A Table showing % change from baseline in EASI score at Day 29 for EES.

FIG. 3B Graph showing % change from baseline in EASI score at Day 29 for EES (ASLAN004 200 mg, 400 mg and 600 mg)

FIG. 4A Graph showing % change in EASI score over time for EES (ASLAN004 200 mg, 400 mg and 600 mg)

FIG. 4B Graph showing % change in EASI score over time for EES (ASLAN004 low and high dose)

FIG. 5A Graph showing % change in baseline in EASI score over time for EES (Placebo)

FIG. 5B Graph showing % change in baseline in EASI score over time for EES (ASLAN004 200 mg)

FIG. 5C Graph showing % change in baseline in EASI score over time for EES (ASLAN004 400 mg)

FIG. 5D Graph showing % change in baseline in EASI score over time for EES (ASLAN004 600 mg)

FIG. 6A Table showing Day 57 sensitivity analysis in the modified intention to treat (mITT)

FIG. 6B Graph showing Day 57 sensitivity analysis in the mITT (ASLAN004 200 mg, 400 mg and 600 mg)

FIG. 6C Graph showing Day 57 sensitivity analysis in the mITT (ASLAN004 low and high dose)

FIG. 7A Summary table showing EASI 50, EASI 75, EASI 90 at Day 57 for EES

FIG. 7B Graph showing EASI 50 at Day 57 for EES (ASLAN004 200 mg, 400 mg and 600 mg)

FIG. 7C Graph showing EASI 50 at Day 57 for EES (ASLAN004 low and high dose)

FIG. 7D Graph showing EASI 75 at Day 57 for EES (ASLAN004 200 mg, 400 mg and 600 mg)

FIG. 7E Graph showing EASI 75 at Day 57 for EES (ASLAN004 low and high dose)

FIG. 7F Graph showing EASI 90 at Day 57 for EES (ASLAN004 200 mg, 400 mg and 600 mg)

FIG. 7G Graph showing EASI 90 at Day 57 for EES (ASLAN004 low and high dose)

FIG. 8A Graph showing proportion of patients achieving EASI 50

FIG. 8B Graph showing proportion of patients achieving EASI 75

FIG. 8C Graph showing proportion of patients achieving EASI 90

FIG. 9 Summary table showing proportion of patients achieving EAS ISO, 75, 90 for EES and mITT

FIG. 10A Summary table showing proportion of patients with IGA score of 0 or 1 at Day 57 for ESS

FIG. 10B Graph showing proportion of patients with IGA score of 0 or 1 at Day 57 for ESS

FIG. 10C Graph showing proportion of patients with IGA score of 0 or 1 over time for ESS

FIG. 11 Table showing baseline TARC and IgE levels of patients

FIG. 12A Graph showing average % change from baseline TARC (ASLAN004 200 mg and 400 mg)

FIG. 12B Graph showing average % change from baseline TARC (ASLAN004 400 mg and placebo)

FIG. 12C Graph showing % change from baseline TARC for individual patients (ASLAN004 400 mg)

FIG. 13A Graph showing IgE % change from baseline for ASLAN004 200 mg and 400 mg

FIG. 13B Graph showing average IgE% change from baseline for ASLAN004 200 mg and 400 mg

FIG. 13C Graph showing IgE % change from baseline for individual patients for placebo

FIG. 13D Graph showing IgE % change from baseline for individual patients for ASLAN004 200 mg

FIG. 13E Graph showing IgE % CFB for individual patients for ASLAN004 400 mg

FIG. 14 Graph showing comparison between ASLAN004 exposure, EASI, TARC and IgE over time for patients receiving ASLAN004 400 mg

FIG. 15 Table showing comparison in activity between ASLAN004 and Duplilumab

FIG. 16 Flow chart showing number of subjects in each test group

FIG. 17 Table showing baseline demographics and disease characteristics of Intention-to-treat (ITT), modified Intention-to-treat (mITT) and Excluded site groups

FIG. 18 Table showing adverse events (AEs) for mITT vs Excluded site groups

FIG. 19 Graph showing EASI, mean % change from baseline (%CFBL) for mITT group

FIG. 20 Graph showing EASI, mean % change from baseline (%CFBL) at week 8 for mITT vs Excluded site groups

FIG. 21 Graph showing placebo adjusted means (EASI) for mITT vs Excluded site groups

FIG. 22 Graph showing % of patients achieving EASI-50, EASI-75 and EAS-90 at week 8 for mITT group

FIG. 23 Graph showing % of patients achieving IGA 0/1 at week 8 for mITT group

FIG. 24 Graph showing mean % CFBL in % BSA affected for mITT group

### EXAMPLES

### Example 1 Study Protocol (Initial MAD Escalation)

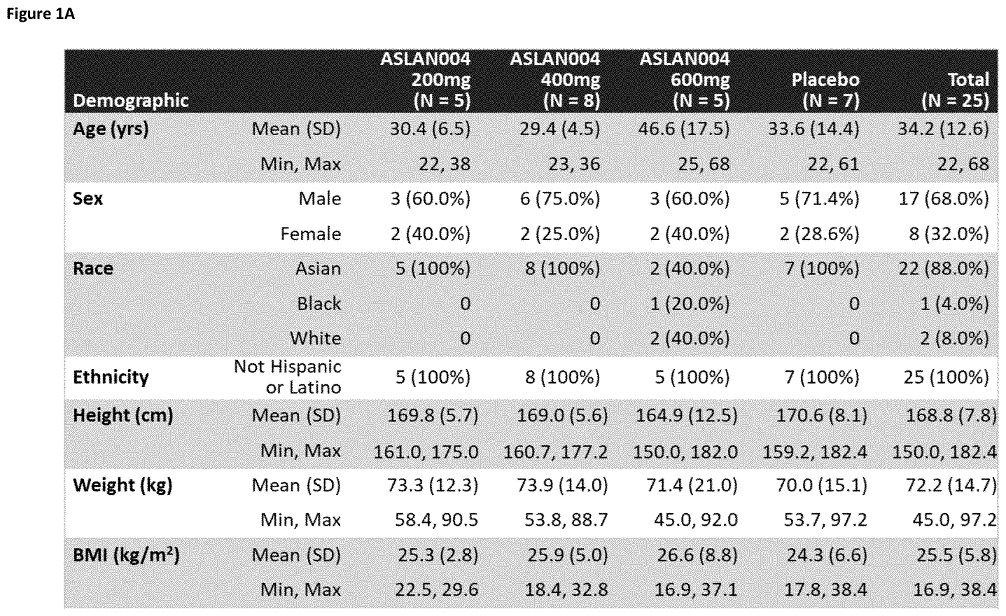

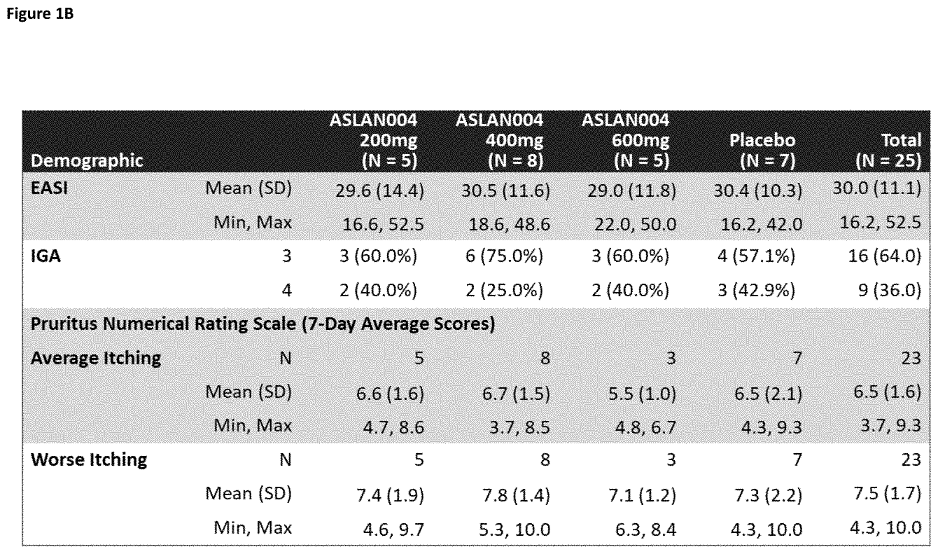

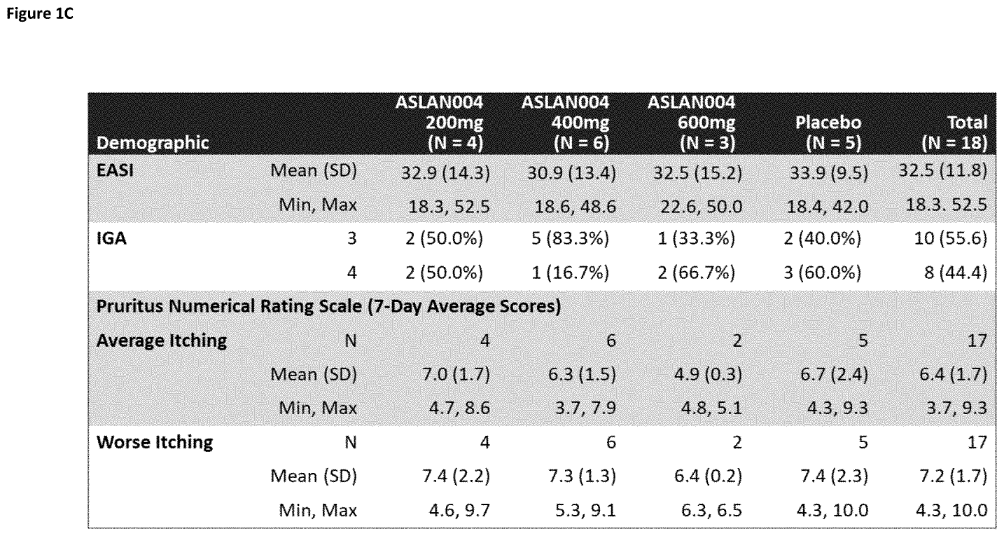

Patients enrolled in ascending dose cohorts of ASLAN004 (SEQ ID NO: 51, 53 and 59 herein): 200 mg 400 mg, 600 mg. ASLAN low dose=ASLAN004 200 mg ASLAN high dose=ASLAN004 400 mg+ASLAN004 600 mg.
Details of patients are shown in FIG. 1. Initially the doses were given weekly (QW). Within each cohort, patients were randomized in a 3:1 ratio of ASLAN004: Placebo

### Results

Table 1 shows the % change in baseline in EASI score at Day 57 (8 weeks).

TABLE 1
% change in baseline in EASI score at Day 57 (8 weeks)

| | | | Diff (vs. |
|---|---|---|---|
| Treatment Arm | N | Mean | Placebo) |
| Placebo | 5 | −42.4% | — |
| ASLAN004 200 mg QW | 4 | −49.5% | −7.1% |
| ASLAN004 400 mg QW | 6 | −73.6% | −31.2% |
| ASLAN004 600 mg QW | 3 | −75.8% | −33.4% |
| ASLAN004 High Doses | 9 | −74.3% | −31.9% |

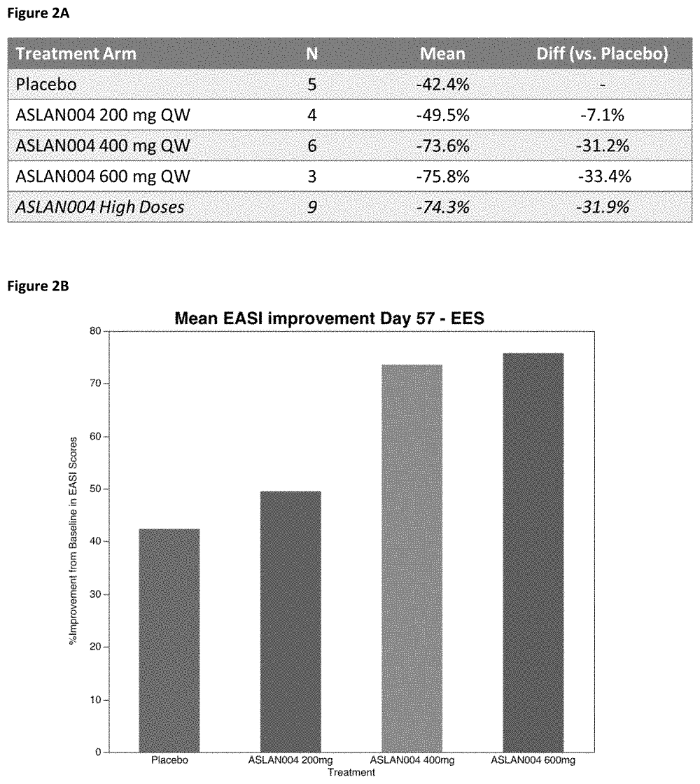

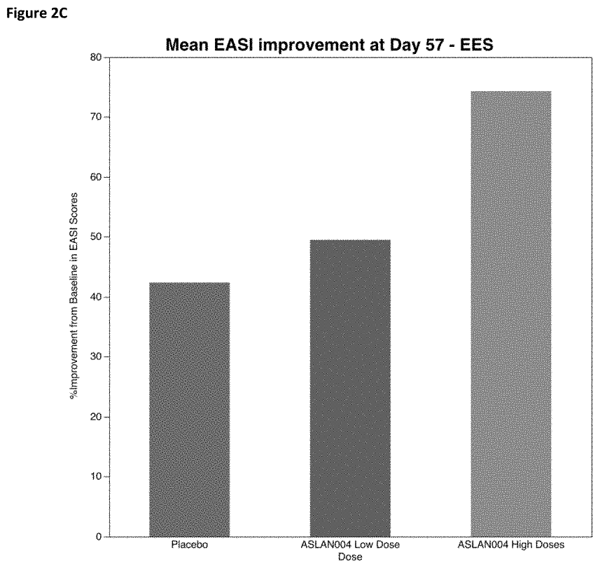

FIGS. 2B to 2C shows the %change in baseline in EASI score in graph form.
Table 2 shows the % change in baseline in EASI score at Day 29 (4 weeks).

TABLE 2
% change in baseline in EASI score at Day 29 (4 weeks)

| | | | Diff (vs. |
|---|---|---|---|
| Treatment Arm | N | Mean | Placebo) |
| Placebo | 5 | −30.5% | — |
| ASLAN004 200 mg | 4 | −51.2% | −20.7% |
| ASLAN004 400 mg | 6 | −63.7% | −33.2% |
| ASLAN004 600 mg | 3 | −53.6% | −23.1% |
| ASLAN004 High Doses | 9 | −60.4% | −29.8% |

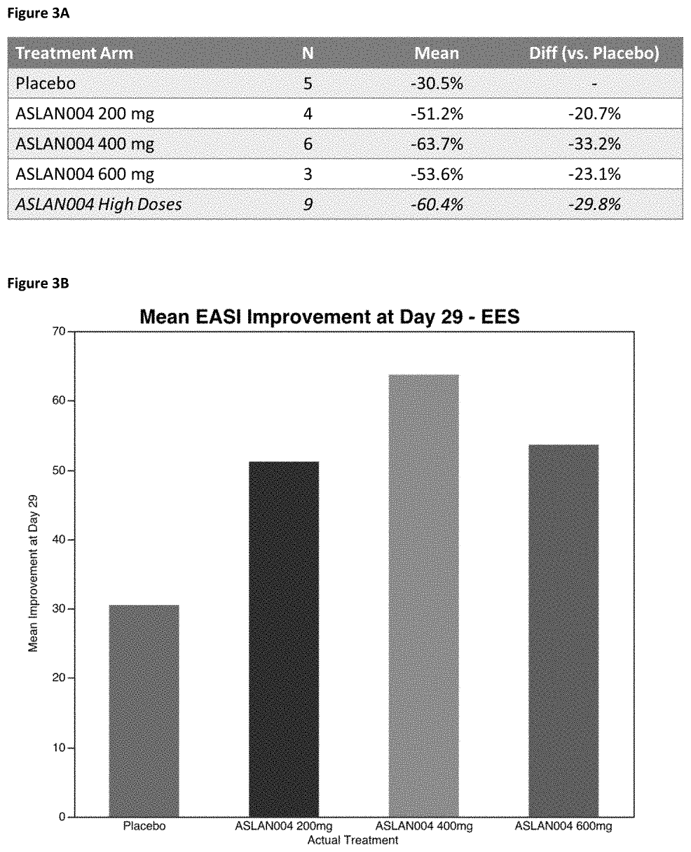

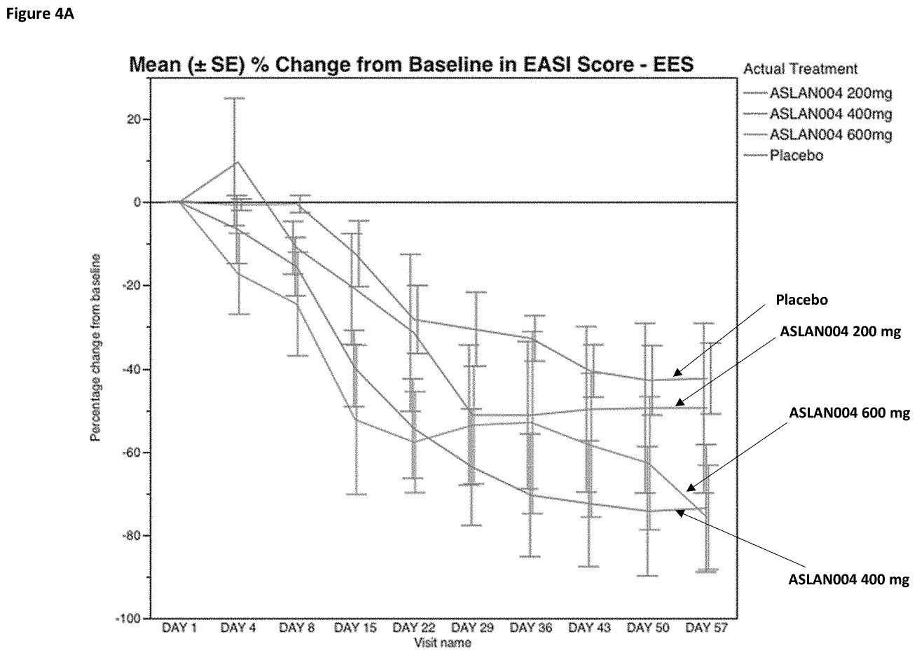

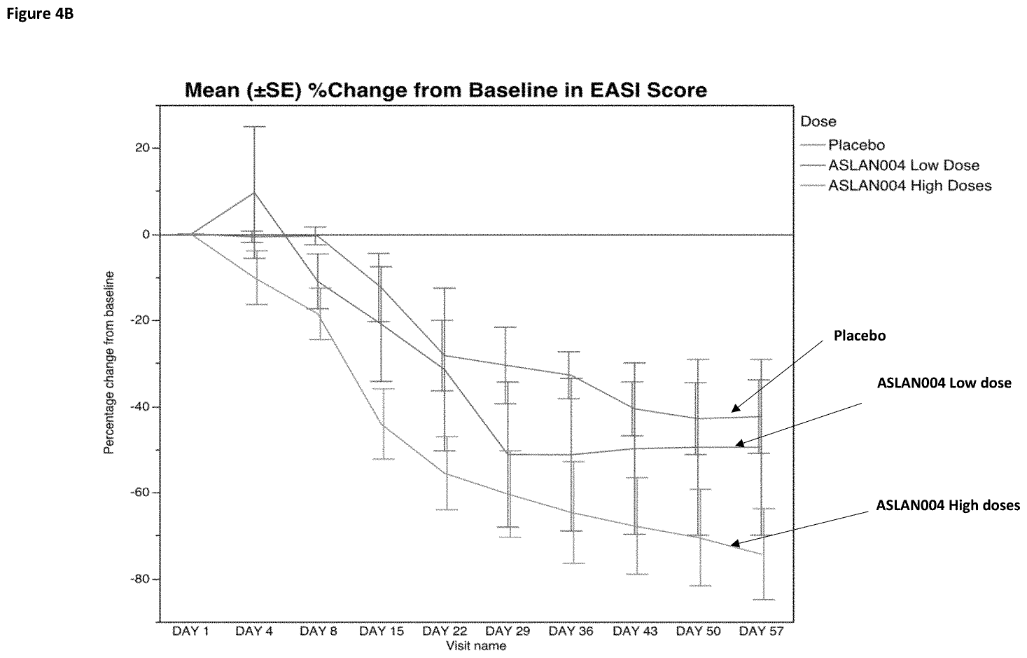

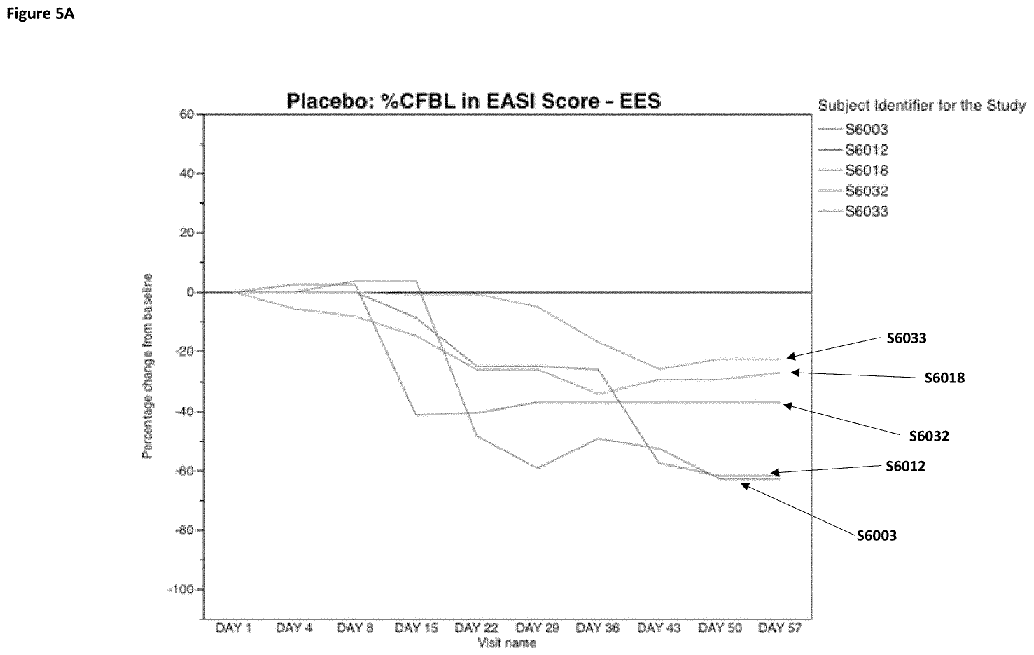

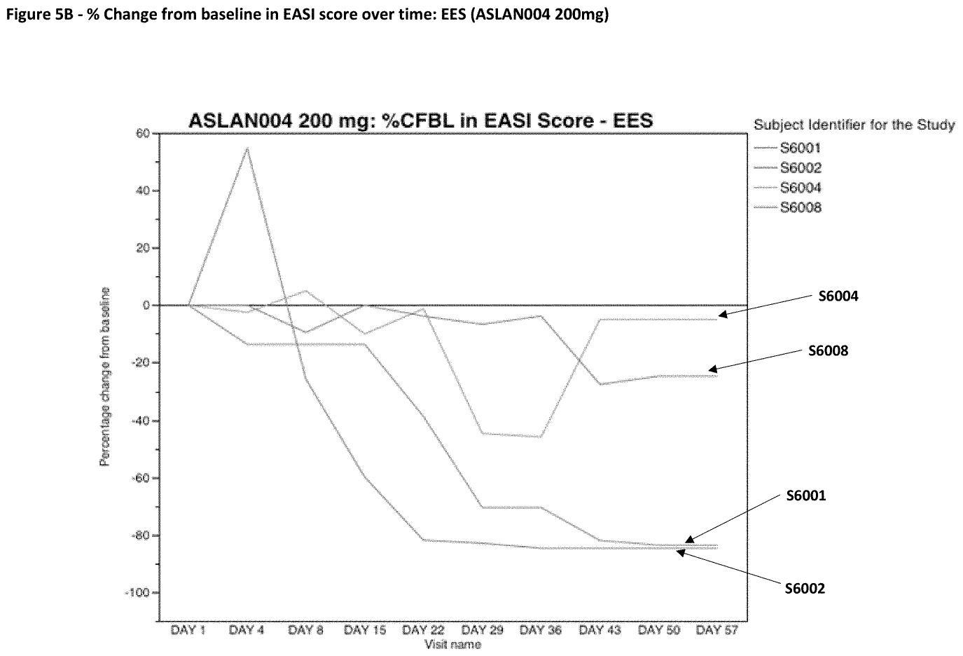

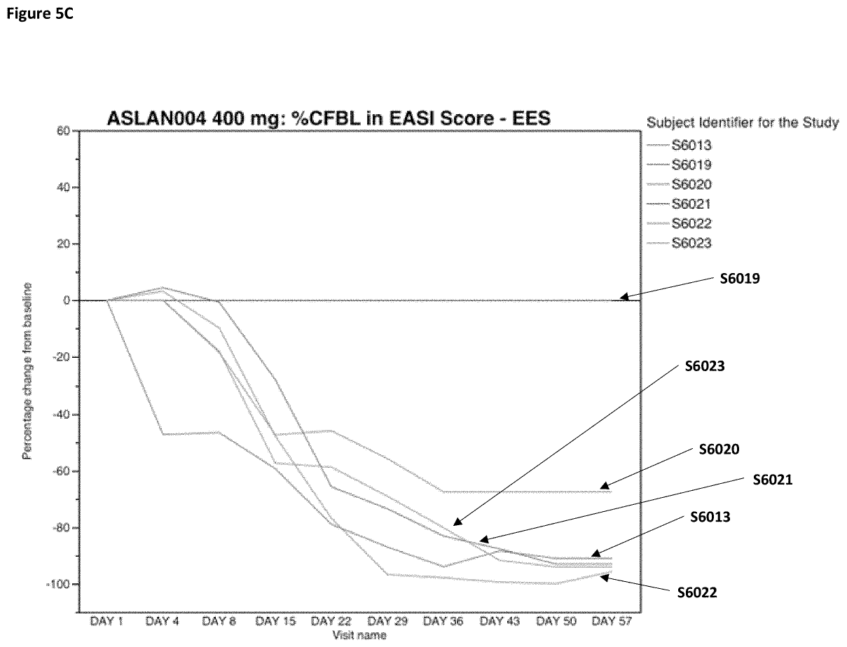

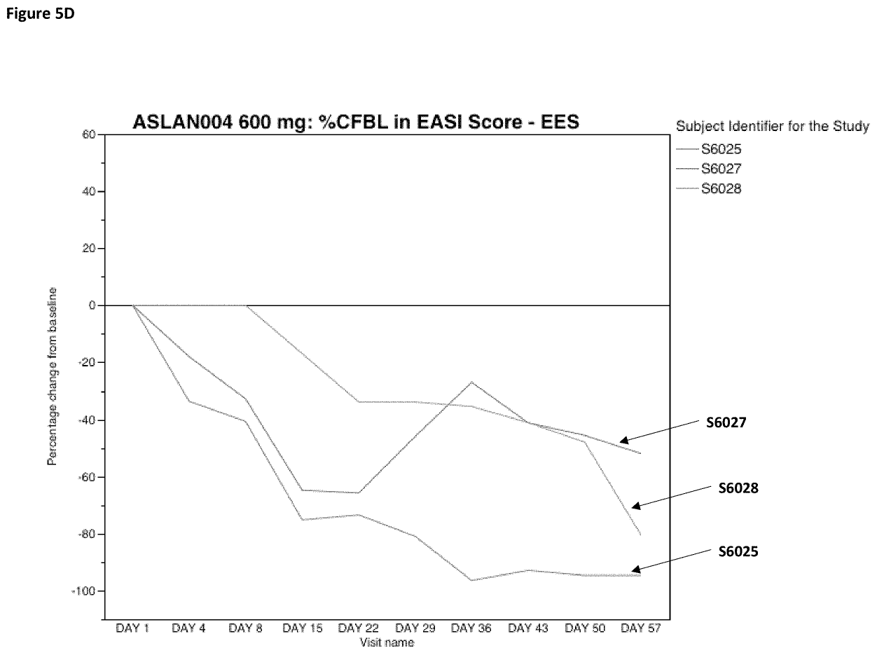

FIG. 3B shows the %change in baseline in EASI score in graph form.
FIGS. 4 and 5 show the % change in baseline in EASI score over time.
Table 3 shows the sensitivity analysis in the mITT (modified intention to treat) set at Day 57

TABLE 3
Sensitivity analysis in the mITT at Day 57

| | | | | Diff (vs. |
|---|---|---|---|---|
| Analysis Set | Treatment Arm | N | Mean | Placebo) |
| EES | Placebo | 5 | −42.9% | — |
| (Primary) | ASLAN004 200 mg | 4 | −49.5% | −7.1% |
| | ASLAN004 400 mg | 6 | −73.6% | −31.2% |
| | ASLAN004 600 mg | 3 | −75.8% | −33.4% |
| mITT | Placebo | 6 | −31.4% | — |
| (sensitivity) | ASLAN004 200 mg | 4 | −49.5% | −18.9% |
| | ASLAN004 400 mg | 7 | −63.1% | −31.6% |
| | ASLAN004 600 mg | 5 | −47.1% | −15.7% |

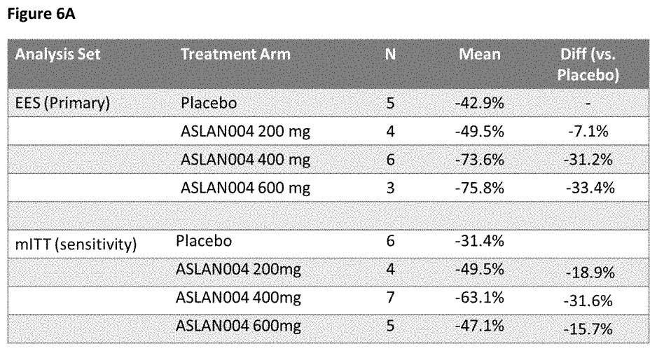

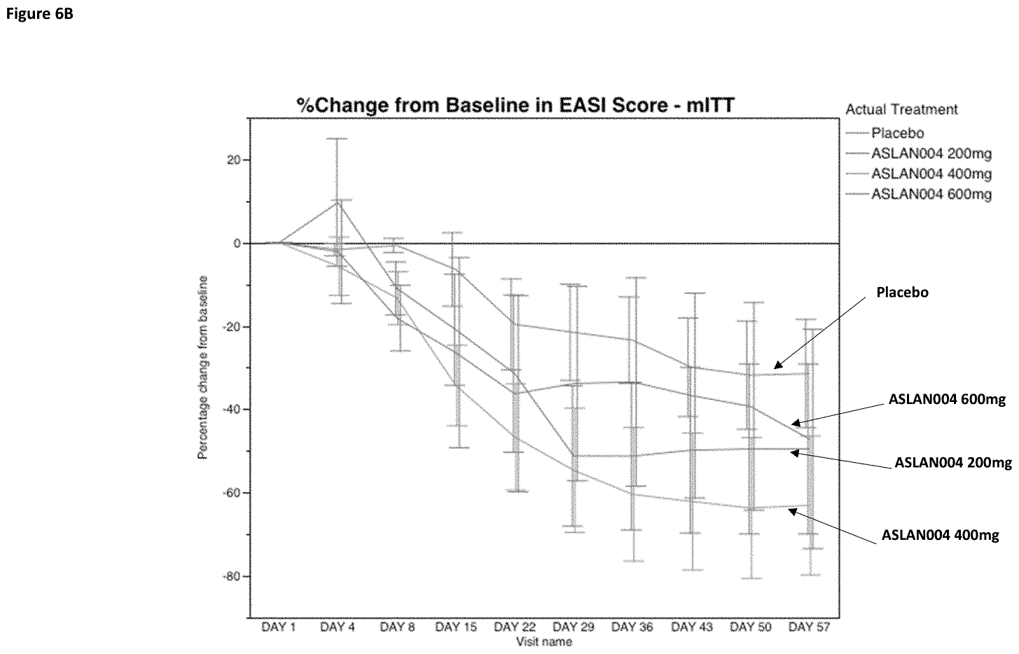

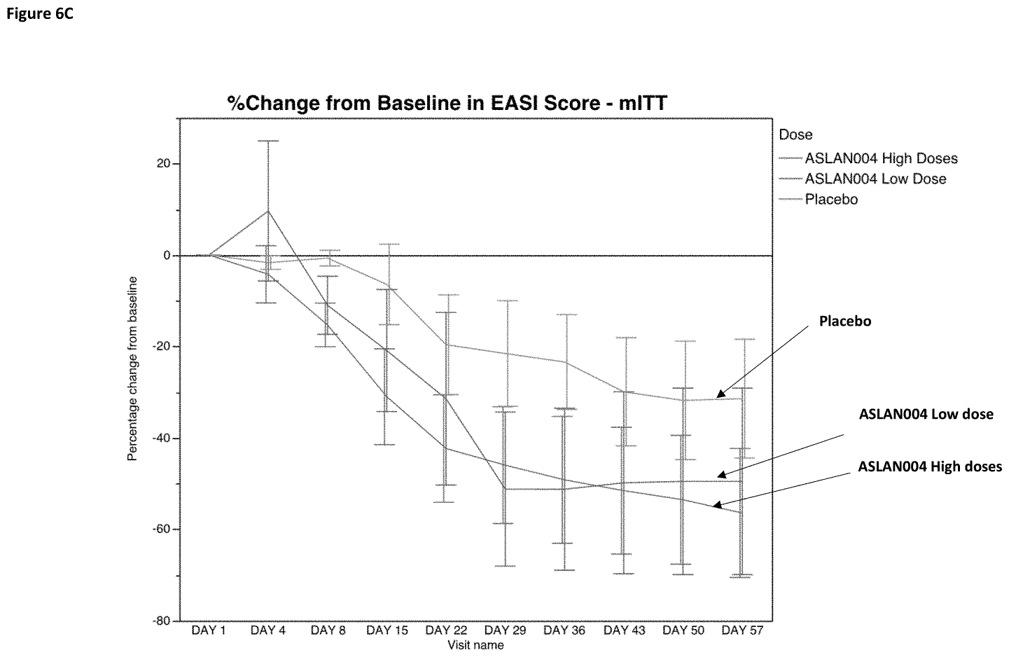

FIG. 6B to C shows the sensitivity analysis in graph form.
Table 4 shows a summary of the proportion of patients achieving EASI 50, EASI 75 and EASI 90 at Day 57.

TABLE 4
EASI 50, EASI 75 and EASI 90 at Day 57

| | EASI 50 | EASI 75 | EASI 90 |
|---|---|---|---|
| Treatment Group | N (%) | N (%) | N (%) |
| Placebo (n = 5) | 2 | 0 | 0 |
| | (40.0%) | | |
| ASLAN004 200 | 2 | 2 | 0 |
| mg (n = 4) | (50.0%) | (50.0%) | |
| ASLAN004 400 | 5 | 4 | 4 |
| mg (n = 6) | (83.3%) | (66.7%) | (66.7%) |
| ASLAN004 600 | 3 | 2 | 1 |
| mg (n = 3) | (100%) | (66.7%) | (33.3%) |
| ASLAN004 High | 8 | 6 | 1 |
| Doses (n = 9) | (88.9%) | (66.7%) | (55.6%) |

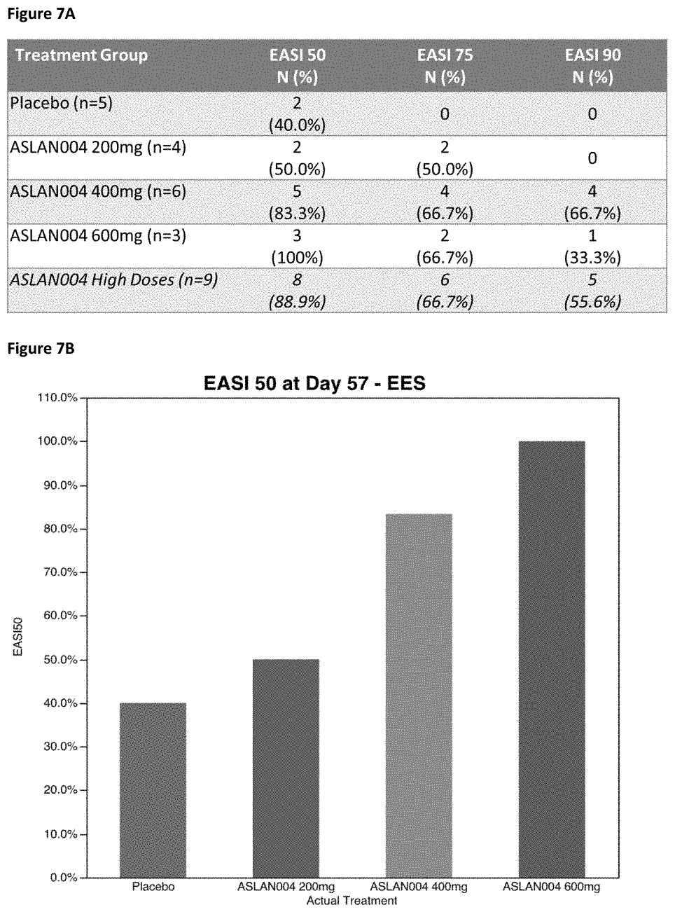

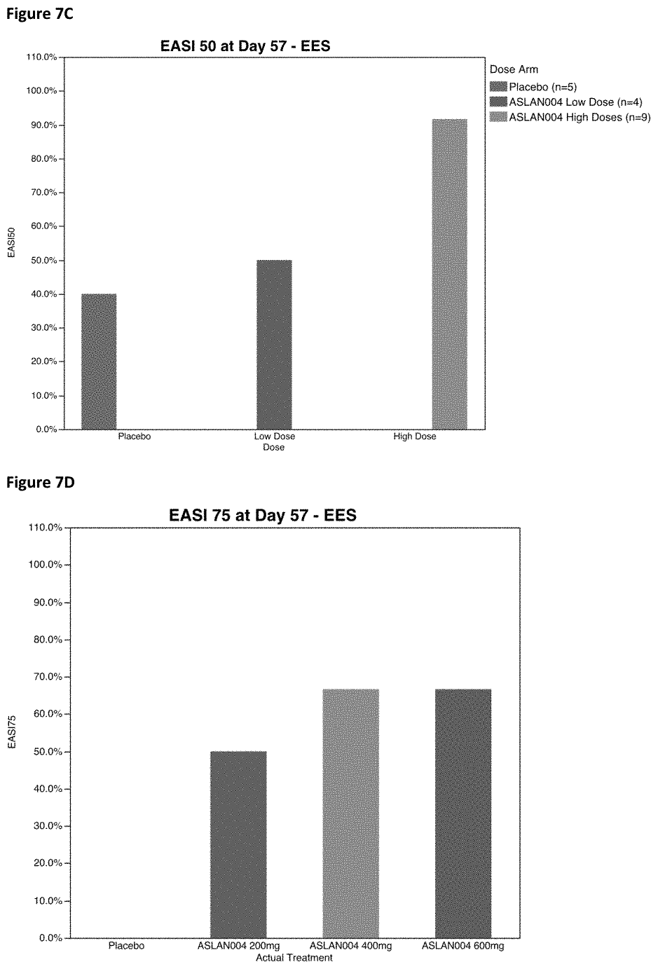

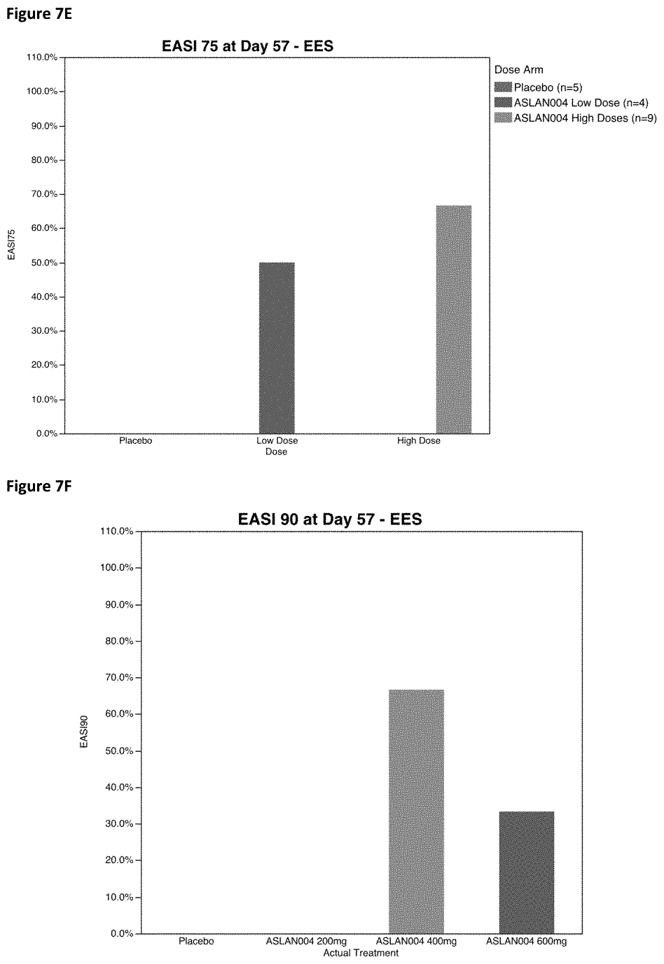

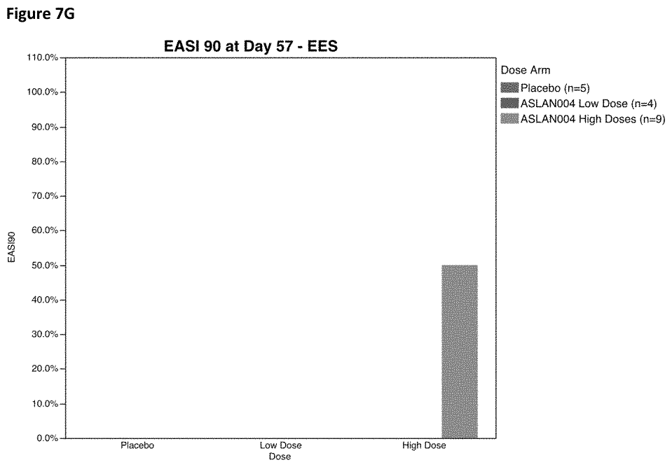

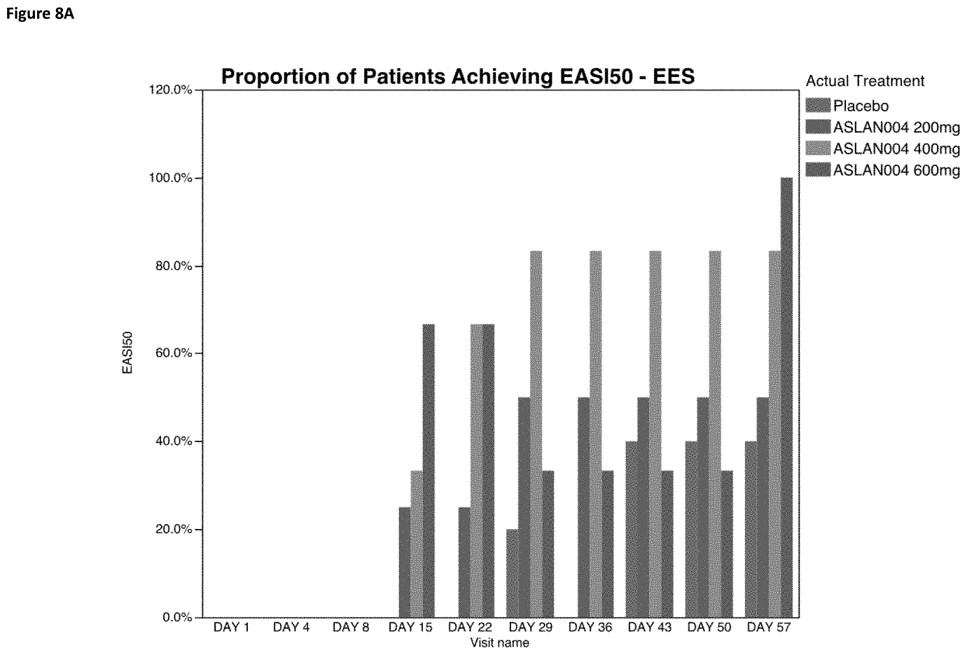

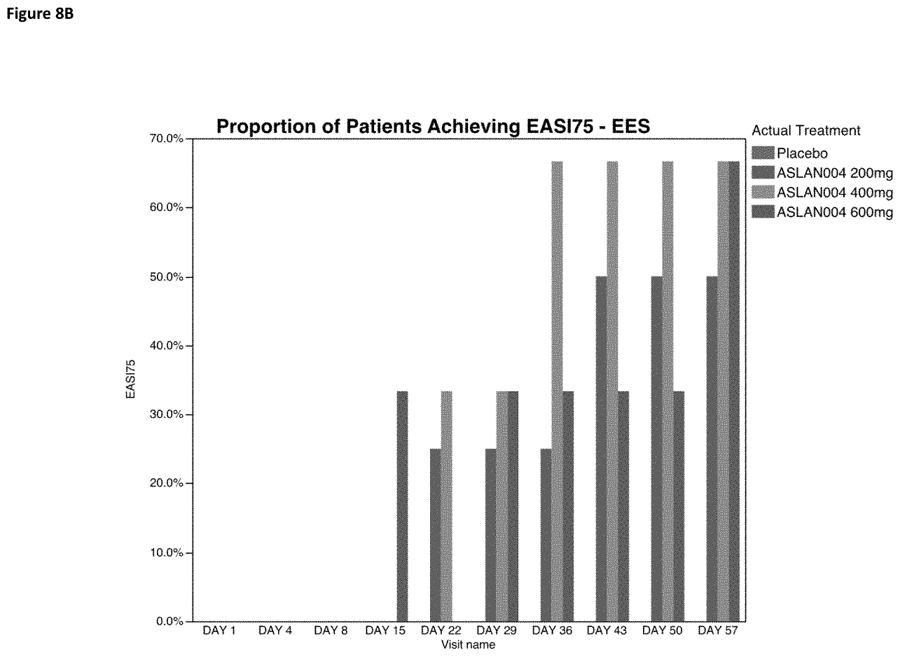

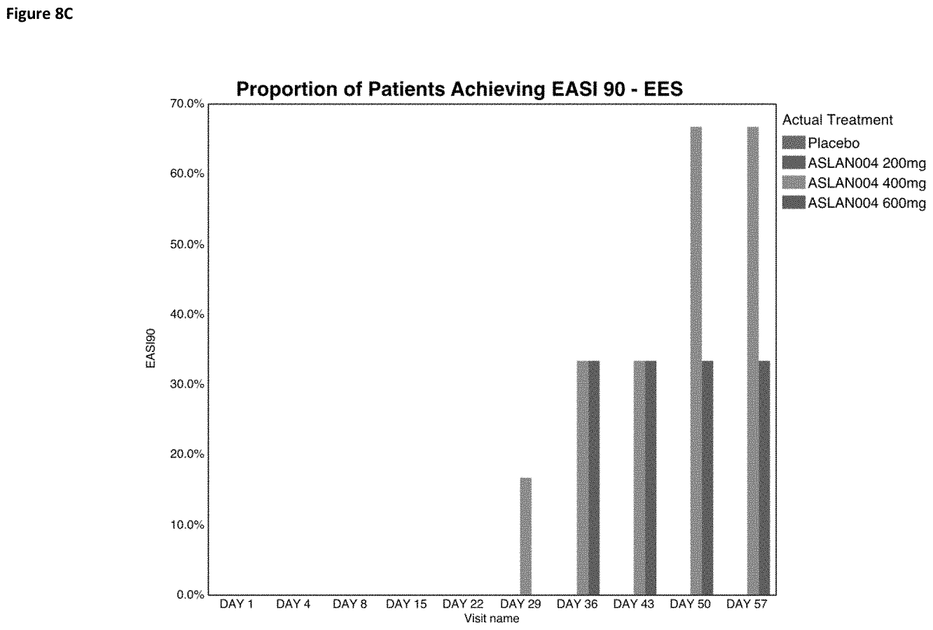

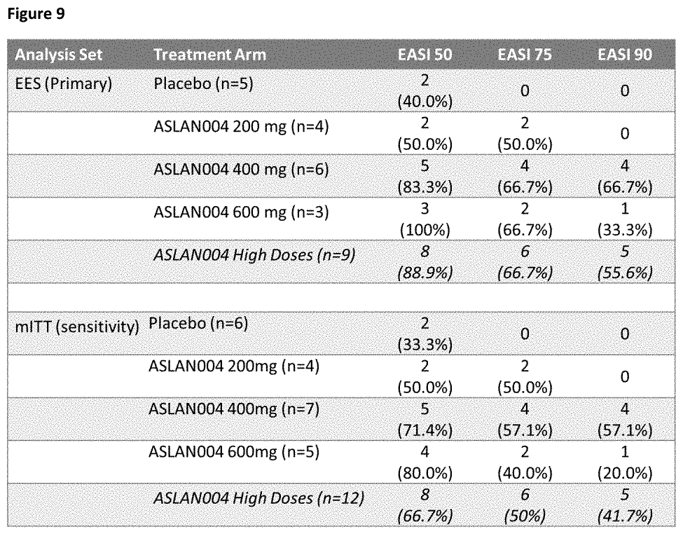

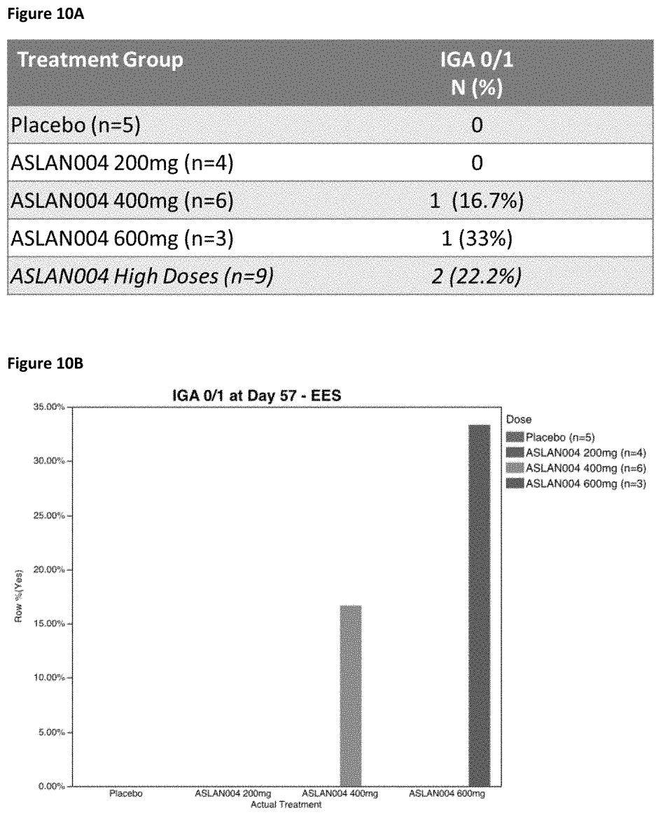

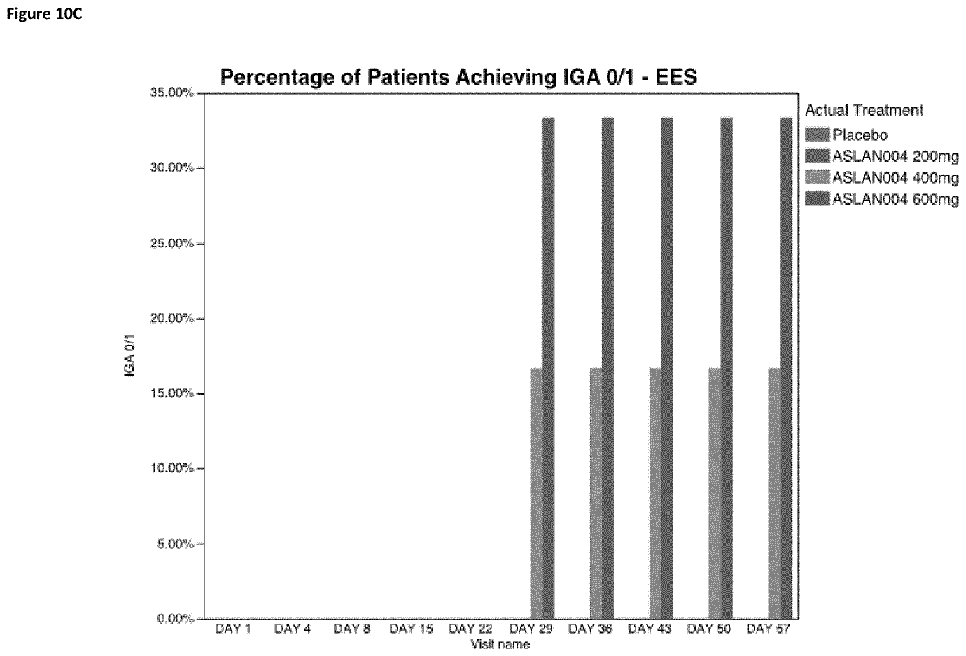

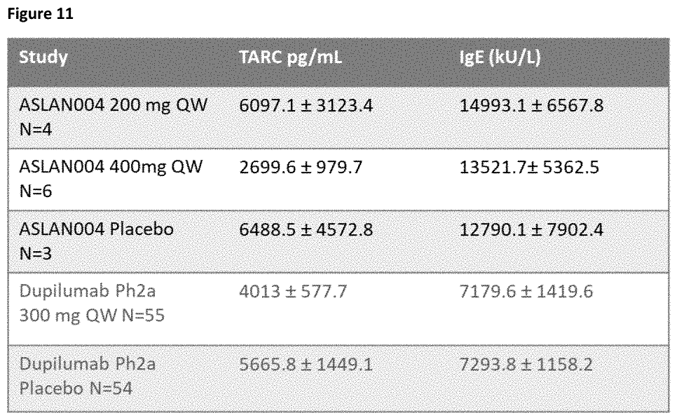

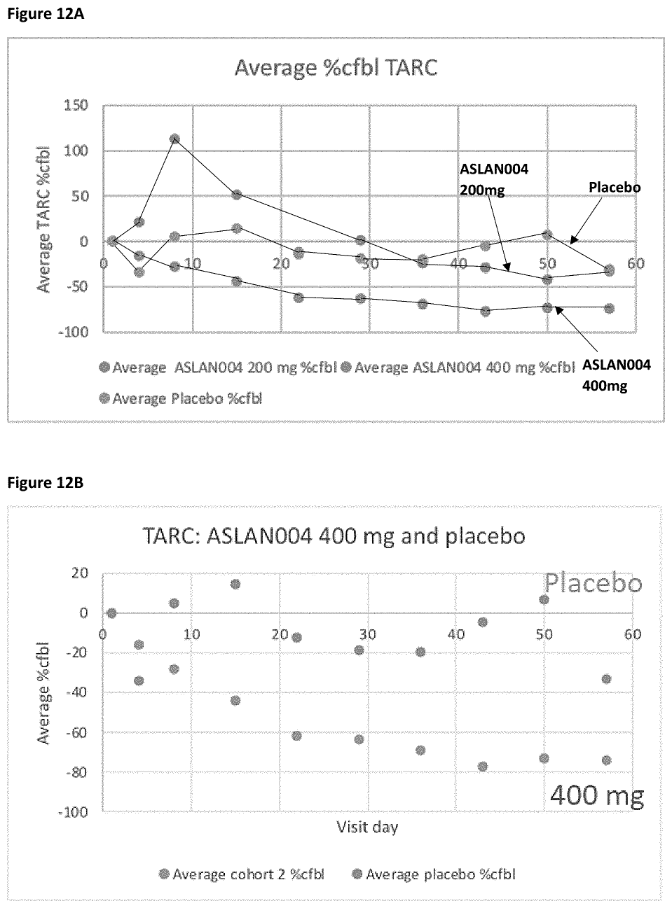

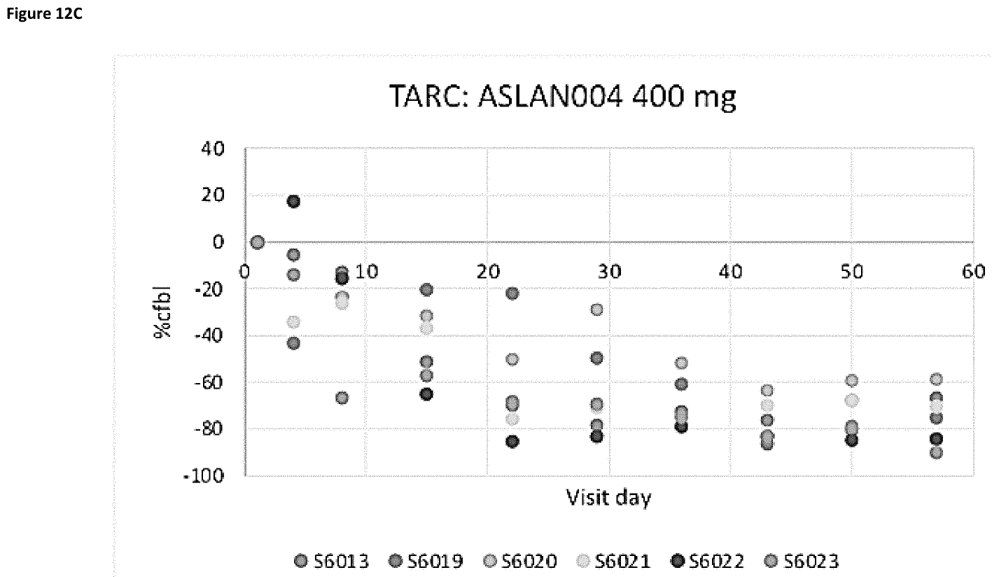

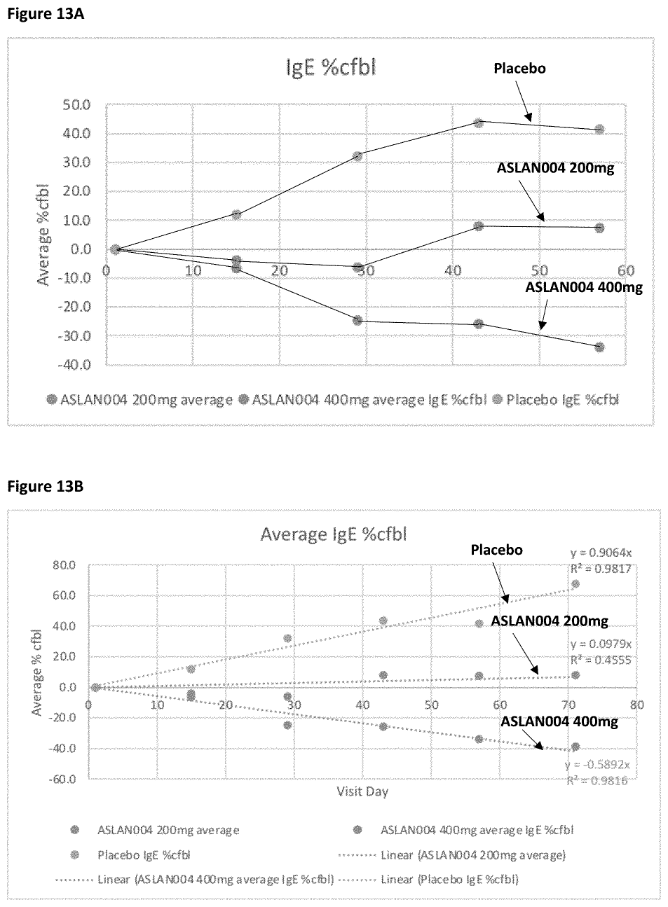

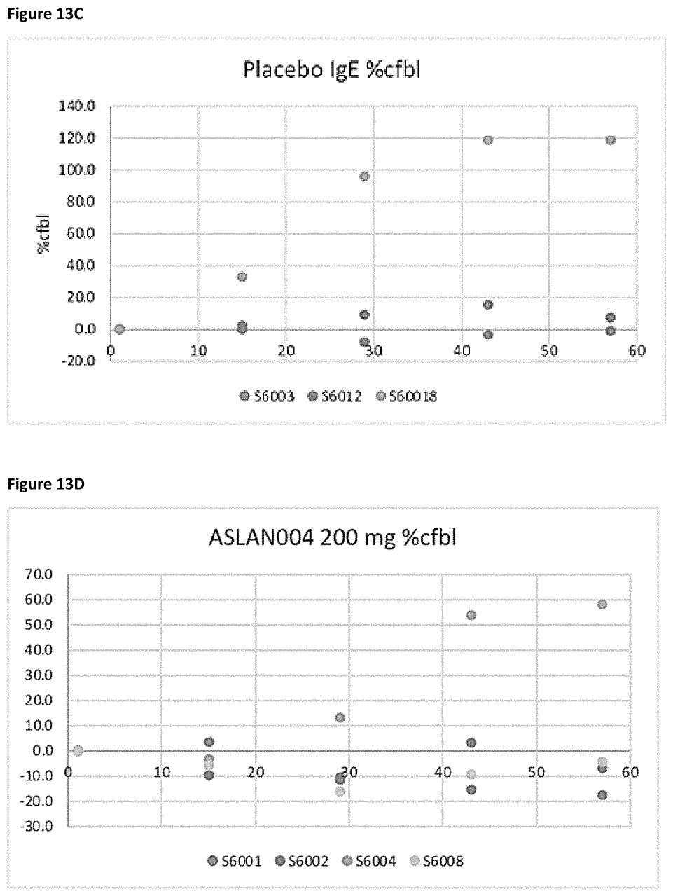

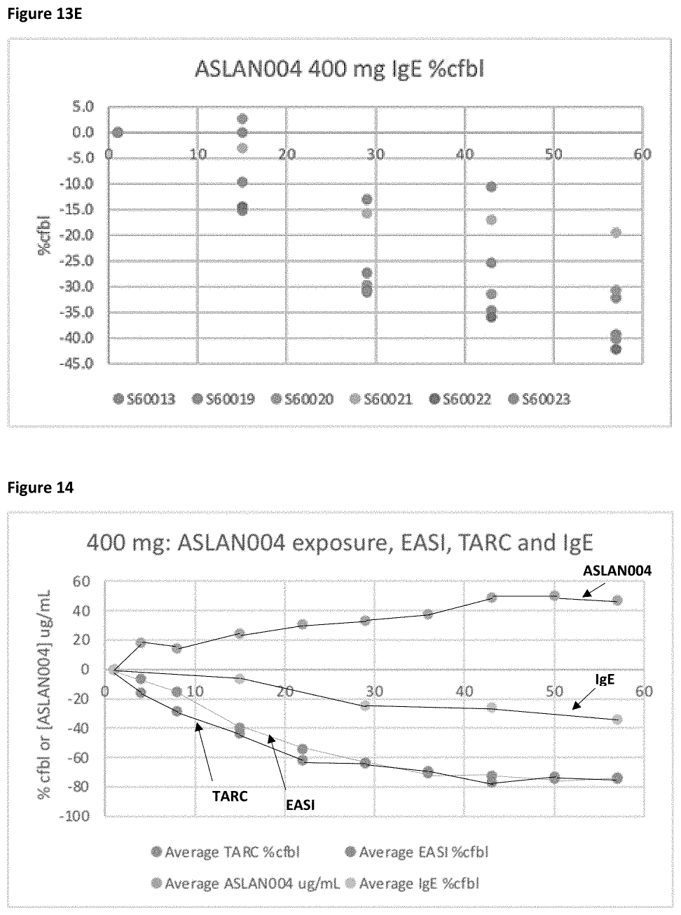

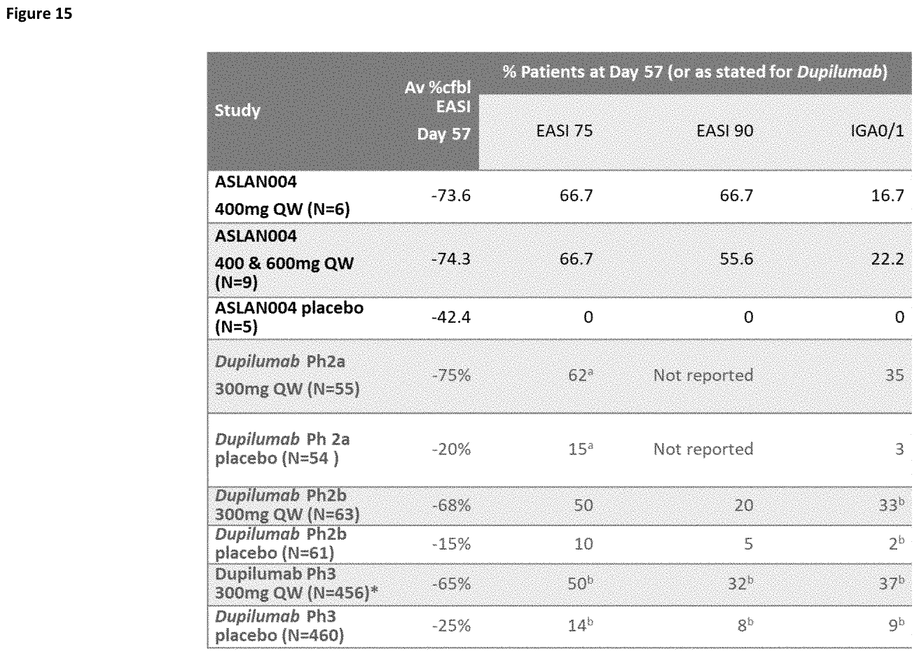

FIGS. 7B to 8C shows the same data in graph form.
FIG. 9 shows the proportion of miTT sensitivity analysis set achieving EASI 50, EASI 75 and EASI 90 at Day 57.
Finally, FIG. 15 shows a comparison between the proportion of patients achieving EASI 50, EASI 75 and EASI 90 at Day 57 when treated with ASLAN004 vs dupilumab.
The results indicate that ASLAN004 results in a significant improvement in EASI score compared to placebo. In particular, at week 8, the average reduction in EASI from baseline at therapeutic doses (400 mg and 600 mg cohorts) was 74% (n=9) compared to 42% (n=5) for patients on placebo.
89% achieved EASI-50 versus 40% on placebo;
67% achieved EASI-75 versus 0% on placebo;
56% achieved EASI-90 versus 0% on placebo
   The results further suggest that ASLAN004 has a comparable or in some cases a higher efficacy compared to Dupilumab, thus demonstrating the potential of ASLAN004 as an alternative therapy for the treatment and management of atopic dermatitis.

### Example 2

In the 32 patients that completed at least 29 days of dosing across all sites, defined in the protocol as the efficacy evaluable data set, the average reduction from baseline in EASI at 8 weeks was 73% (n=19) compared to 44% (n=13) for patients on placebo (p=0.007¹).

The proportion of patients with adverse events and treatment-related adverse events were similar across treatment and placebo arms. There were no incidences of conjunctivitis in the expansion cohort.

TABLE 5
EASI score for RITT and ITT groups

| | RITT (n = 29) | | | ITT (n = 38) | | |
|---|---|---|---|---|---|---|
| | 600 mg | Placebo | | 600 mg | Placebo | |
| Endpoint | (n = 16) | (n = 13) | p-value¹ | (n = 22) | (n = 16) | p-value¹ |
| Mean % change from | −64.9 | −27.2 | 0.021 | −61.3 | −31.9 | 0.023 |
| baseline in EASI | | | | | | |
| EASI-50 (%) | 81.3 | 30.8 | 0.008 | 77.3 | 37.5 | 0.016 |
| EASI-75 (%) | 68.8 | 15.4 | 0.005 | 50.0 | 12.5 | 0.018 |
| EASI-90 (%) | 37.5 | 15.4 | 0.183 | 27.3 | 12.5 | 0.245 |
| IGA 0/1 (%) | 43.8 | 15.4 | 0.107 | 31.8 | 18.8 | 0.301 |
| Mean % change from | −38.6 | −15.3 | 0.051 | −37.1 | −15.7 | 0.032 |
| baseline in peak pruritis | | | | | | |
| Numerical Rating Scale | | | | | | |
| Mean change from | −9.8 | −2.5 | 0.007 | −9.0 | −3.5 | 0.014 |
| baseline in POEM | | | | | | |

¹One-sided p-value

ASLAN004 achieved a statistically significant improvement (p<0.025) versus placebo in the primary efficacy endpoint of percent change from baseline in the Eczema Area Severity Index (EASI), and also showed significant improvements (p<0.05) in other key efficacy endpoints: EASI-50, EASI-75, peak pruritis and the Patient-Oriented Eczema Measure (POEM).

Following discussions with the Data Monitoring Committee prior to unblinding, a Revised ITT population (RITT, n=29) was defined to exclude one study site at which all patients enrolled in the study appeared atypical of moderate-to-severe AD patients based on biomarkers, such as TARC, and patient medical history. In the RITT population, which is more comparable to other published studies in moderate-to-severe AD, ASLAN004 also achieved a statistically significant improvement (p<0.025) versus placebo in percent change from baseline in EASI and showed a greater improvement over placebo in the key efficacy endpoints versus the ITT population.

### Example 3

The objective of this study was to further analyze secondary endpoints of clinical relevance and post hoc subgroup analyses.

### Methods

Three patient cohorts were randomized to receive either 200, 400 or 600 mg eblasakimab or placebo subcutaneously once weekly for 8 weeks in a multiple ascending dose study design.

Adult patients were included with chronic AD present for years before screening, and the following atopic dermatitis (AD) parameters at screening and baseline: eczema area and severity index (EASI) 16, Investigator's Global Assessment (IGA) score (scale of 0 to 4), and 10% body surface area (BSA) of AD involvement. Rescue medication (moisturizer with active ingredient, topical corticosteroids, topical calcineurin inhibitors) was not allowed; LOCF was used for participants who used rescue med.

Efficacy assessments included percent change from baseline (%CFBL) in EASI, proportions of patients with 50% or 75% improvement in EASI score (EASI 50 or EASI 75) or IGA 0/1, and % CFBL in percent BSA involvement. Further data are presented from a prespecified subgroup of patients from an excluded site with atypical AD.

Inferential statistical analysis was performed for 600 mg vs. placebo groups at week 8 only; results for 200 and 400 mg groups were descriptively described due to small sample size.

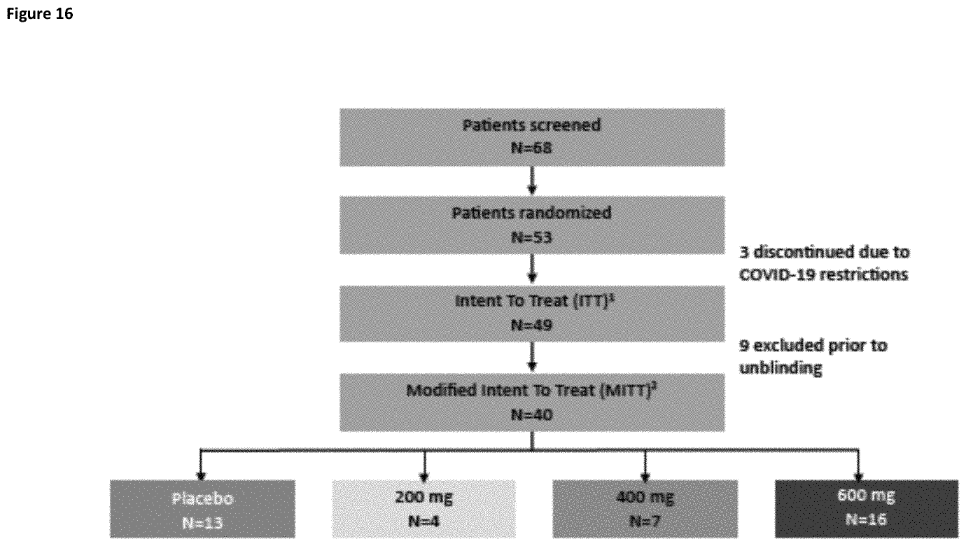

Efficacy analysis in the Phase 1b study used a modified Intent to Treat (mITT) population in which 9 study patients from one site were excluded (excluded site group) from the ITT analysis prior to unblinding as the participants did not have disease characteristics consistent with moderate to severe AD (FIG. 16).

### Results

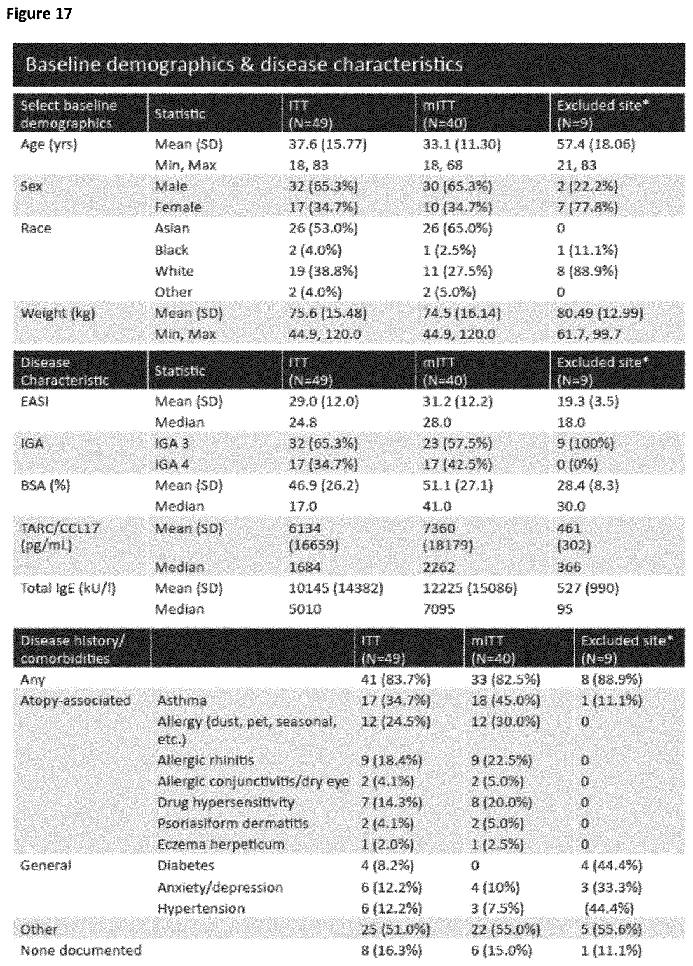

The Excluded site set was markedly different from the mITT set at baseline with substantially lower serum TARC/CCL17 (7,350 pg/mL and 461 pg/mL, respectively), serum IgE (12,225 kU/I vs 527 kU/I), and EASI scores (mean 31.2 vs 19.3) showing lower extent and severity of disease. Other notable differences included older age, and lower IGA and BSA. Participants in this site had no atopic disease history but reported other comorbidities including diabetes and hypertension (FIG. 17).

In the mITT analysis set, improvements in EASI score were seen early and progressed over the trial duration with eblasakimab treatment compared with placebo, with the 400 and 600 mg doses producing a great magnitude of response than the 200 mg dose (FIG. 19).

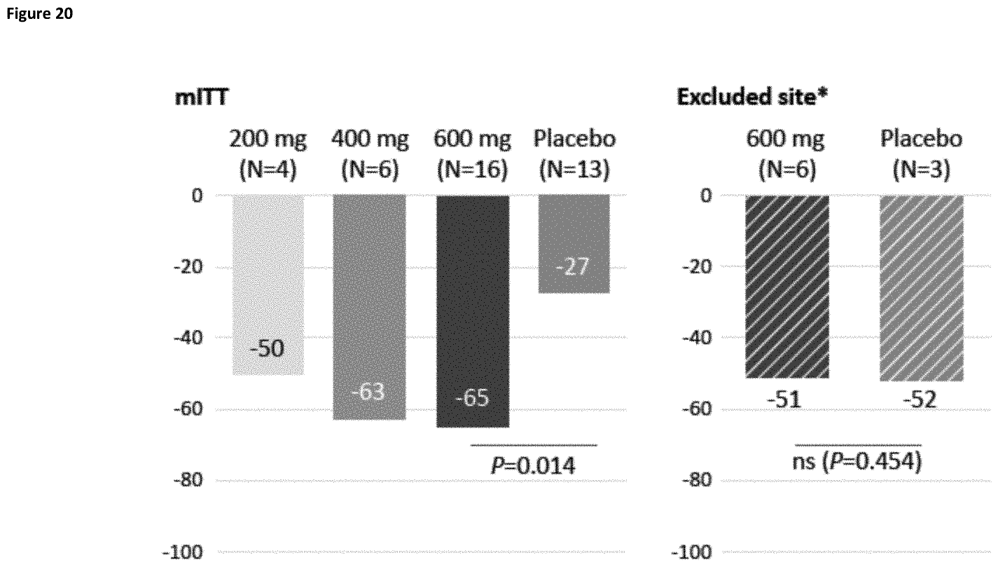

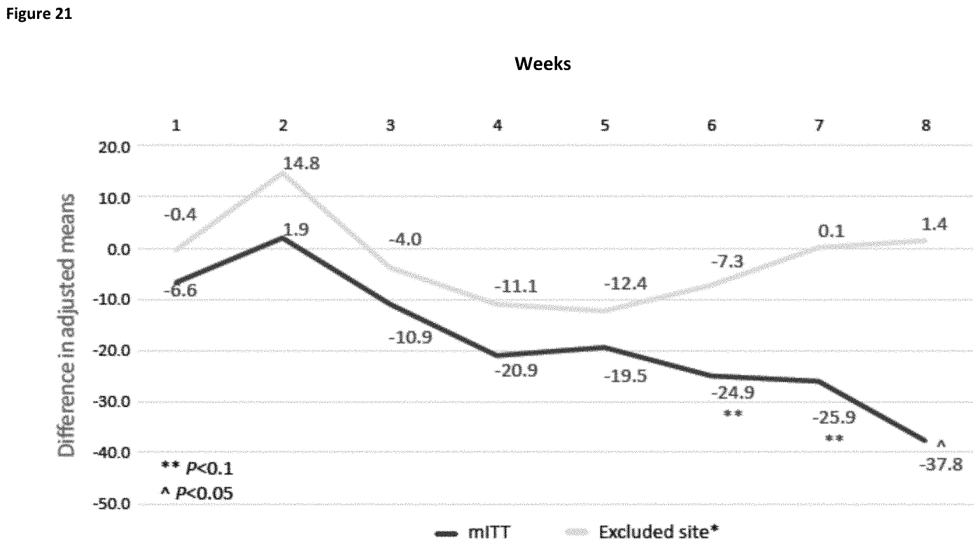

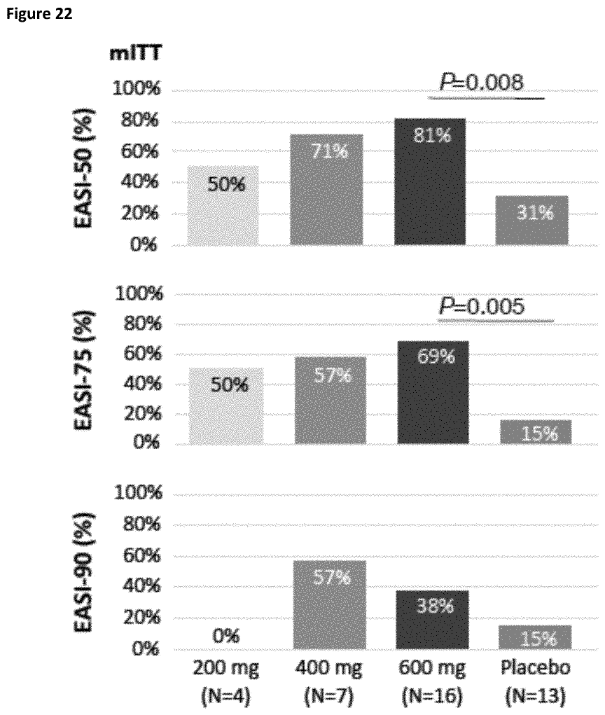

Significant improvements in %CFBL in EASI score at week 8 were noted for eblasakimab 600 mg vs. placebo in the mITT set (−65% vs. −27%, P=0.014), but not the Excluded site population (FIG. 20). The difference in adjusted means was apparent early for the mITT set by week 6 but not for the Excluded site set (FIG. 21).

Though the study was not powered to achieve statistical significance in any of the binary outcomes, improvements were nonetheless observed in the mITT set in the proportions of patients taking eblasakimab who achieved EASI-50, EASI-75 and EASI-90 over time vs. placebo at week 8 (EASI-50: 81% vs. 31%; EASI-75: 69% vs. 15%; EASI-90: 38% vs. 15%) (FIG. 4). No such improvement was apparent in the Excluded site*population.

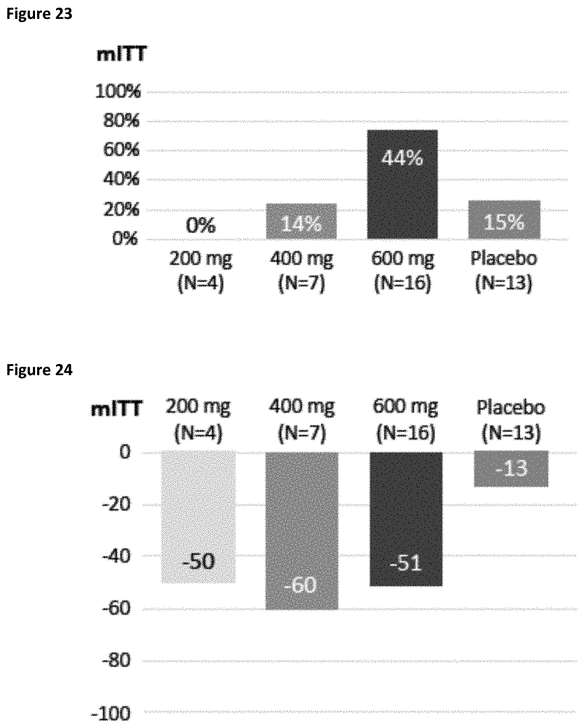

A higher percentage of patients achieved an IGA 0/1 at week 8 for eblasakimab 600 mg vs placebo in the mITT set (44% vs. 15%, P=0.107) but not the Excluded site population (FIG. 23).

Mean % CFBL in BSA at week 8 was −51% for eblasakimab 600 mg vs. −13% for placebo in the mITT set and −39% vs. −44% in the Excluded site population (FIG. 24).

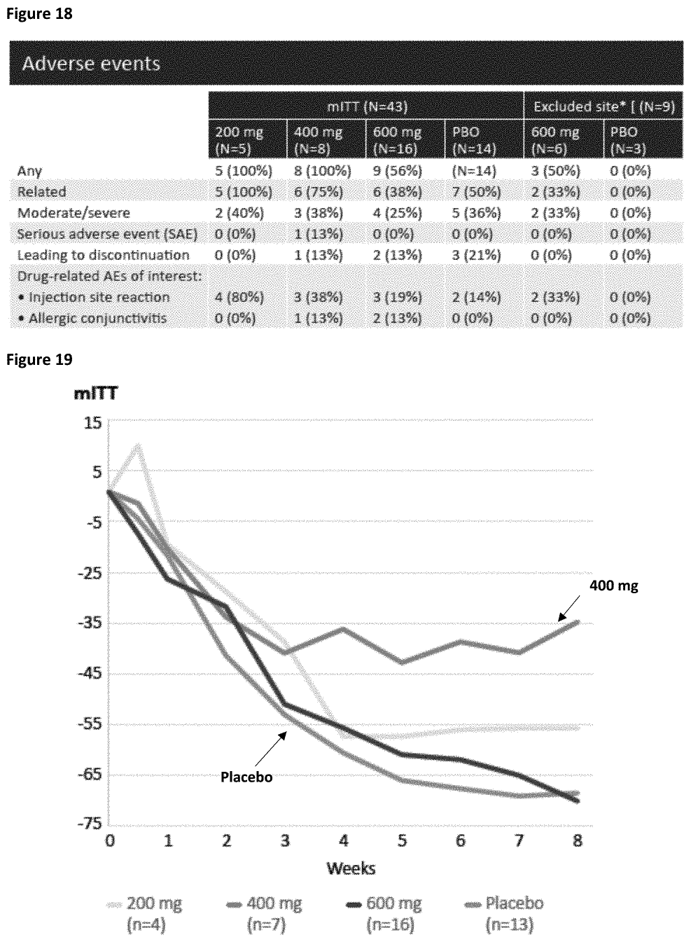

Rescue medication use was low, but higher in the placebo group (data not shown). Rates of moderate-to-severe Adverse events (AEs) were comparable between 600 mg and placebo. AEs related to treatment were similar between groups (FIG. 18).

Interestingly, AEs leading to treatment discontinuation were higher in the placebo group. 1 serious adverse event (SAE) reported in the study (mild abdominal pain, 400 mg); considered unrelated to treatment. No deaths were reported.

### CONCLUSION

This study indicates that eblasakimab was well tolerated with significant improvements vs. placebo in several efficacy outcomes in a Phase 1b study in adults with moderate to severe AD. Robustness of the data from the small study was supported by sensitivity analyses on the primary analysis set Including the Excluded site data did not change the primary endpoint or conclusions.

That these significant improvements were seen within the 8-week study period offers the potential for a greater magnitude of effect with prolonged treatment, supporting further investigation in an ongoing Phase 2b clinical trial.

1. A method of treating a patient to reduce EASI score in the range −20 to −100% from the baseline in a patient with atopic dermatitis, for example moderate to severe atopic dermatitis (in particular poorly controlled moderate to severe atopic dermatitis) by parenteral administration of a treatment cycle comprising a dose in the range 200 mg to 600 mg of an antibody, antigen binding fragment thereof or a pharmaceutical formulation thereof , which is an inhibitor of signalling through of the IL-13Rα1 by binding the said receptor.

2. A method according to claim 1, wherein reduction in EASI is present after about two weeks from administration of the first dose (such as day 15).

3. A method according to claim 1, wherein reduction in EASI is present after about four weeks from administration of the first dose (day 29).

4. A method according to claim 1, wherein reduction in EASI is present after about six weeks from administration of the first dose (such as day 43).

5. A method according to claim 1, wherein reduction in EASI is present after about eight weeks from administration of the first dose (such as day 57).

6. A method according to claim 1, wherein multiple doses are administered in a treatment cycle.

7. A method according to claim 1, wherein multiple treatment cycles are administered, for example 2, 3, 4 or more treatment cycles are administered.

8. A method according to claim 1, wherein following the treatment cycle or cycles and disease modification, maintenance therapy is administered, for example the same dose administered less frequently (for example monthly), or a lower dose (such as 200 mg) administered the same frequency or less frequently (such as about 2 weekly, about 3 weekly, or about 4 weekly.

9. A method according to claim 1, wherein said antibody or binding fragment thereof is administered approximately weekly, once approx. every 2 weeks, once approximately every 3 weeks, or once approximately every 4 weeks (for example monthly), (in particular a single treatment cycle, especially 8 weeks).

10. A method according to claim 1, wherein a loading dose in the range 400 to 900 mg, for example 400, 500, 600, 700, 800 or 900 mg is employed before administration of the treatment cycle.

11. A method according to claim 1, wherein the treatment does not comprise a loading dose.

12. A method according to claim 1, wherein the dose is 200 mg.

13. A method according to claim 12, wherein the reduction in EASI is in the range −15 to −60% (for example at about day 15).

14. A method according to claim 12, wherein the reduction in EASI score is in the range −40 to −85% (eg at about day 29).

15. A method according to claim 12, wherein the reduction in EASI score is in the range −25 to −85% (eg at about day 43 or 57)

16. A method according to claim 12, wherein the dose is in the range 350 to 450 mg, such as 400 mg, for example wherein 80% of the patient population has an EASI 50 at about day 29 and/or day 57.

17. A method according to claim 1, wherein the dose is 600 mg.

18. A method according to claim 17, wherein the reduction in EASI score is in the range −25 to −60% (eg −39 to −59, such as −40 to −59, in particular −47, −48, −49, −50, −51, −52, −53, −54, −55, −56, −57, −58 or −59%) for example at about day 15.

19. A method according to claim 17, wherein the reduction in EASI is in the range −50 to −100% (eg −55 to −97%) such as at about day 29.

20. A method according to claim 17, wherein the reduction in EASI is in the range −60 to −100% (eg −70 to −97%) in particular at about day 43.

21. A method according to claim 17, wherein the reduction in EASI score is in the range −65 to −100% (for example −70 to −100, such as −90 to −100, in particular 91, 92, 93, 94, 95, 96, 97, 98, 99 or 100%) in particular at about day 57.

22. A method according to claim 17, wherein 90% of the patient population has an EASI 50 at about day 57.

23. A method according to claim 1, wherein the treatment cycles comprises a first dose at 600 mg, followed by three weekly doses of 400 mg, e.g. wherein the treatment cycle is repeated twice i.e. two treatment cycles lasting 8 weeks, in particular day 1 600 mg, approx. day 8 400 mg, approx. day 15 400 mg, approx. day 22 400 mg, approx. day 29 600 mg, approx. day 36 400 mg, approx. day 43 400 mg, and approx. day 50 400 mg are administered.

24. A method according to claim 1, wherein disease modification, occurs by day 4, wherein day 1 is the first administration of the antibody or binding fragment thereof, for example wherein the disease modification is a reduction in EASI score, such as wherein the reduction is a percentage from base line in the range −10 to 55%.

25. A method according to claim 1, wherein the disease modification in the range −40 to −100% is achieved by about day 57 following first administration on day 1, e.g. maximum disease modification is achieved by about day 57.


SEQUENCE LISTING (63 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDRH3
  SITE 1: note = MISC_FEATURE - Xaa denotes Phe, Met, Gln, Leu or Val
  SITE 6: note = MISC_FEATURE - Xaa denotes Ser or Ala
  SITE 7: note = MISC_FEATURE - Xaa denotes Phe, Leu, Ala or Met
  SITE 9: note = MISC_FEATURE - Xaa denotes Tyr, Gln, Lys, Arg, Trp, His, Ala, Thr, Ser, Asn orGly
XPNWGXXDX

SEQ ID NO: 4 - protein, 9 aa, synthetic construct
  REGION 1..9: note = CDR H3 SEQUENCE
FPNWGALDQ

SEQ ID NO: 5 - protein, 9 aa, synthetic construct
  REGION 1..9: note = CDR H3 SEQUENCE
VPNMGSLDT

SEQ ID NO: 6 - protein, 9 aa, synthetic construct
  REGION 1..9: note = CDR H3 SEQUENCE
FPNWGSMDA

SEQ ID NO: 7 - protein, 9 aa, synthetic construct
  REGION 1..9: note = CDR H3 SEQUENCE
FPNWGSLDH

SEQ ID NO: 8 - protein, 9 aa, synthetic construct
  REGION 1..9: note = CDR H3 SEQUENCE
MPNWGSFDY

SEQ ID NO: 9 - protein, 9 aa, synthetic construct
  REGION 1..9: note = CDR H3 SEQUENCE
MPNWGSFDT

SEQ ID NO: 10 - protein, 9 aa, synthetic construct
  REGION 1..9: note = CDR H3 SEQUENCE
MPNWGSLDH

SEQ ID NO: 11 - protein, 9 aa, synthetic construct
  REGION 1..9: note = CDR H3 SEQUENCE
MPNWGSFDS

SEQ ID NO: 12 - protein, 9 aa, synthetic construct
  REGION 1..9: note = CDR H3 SEQUENCE
MPNWGSLDT

SEQ ID NO: 13 - protein, 9 aa, synthetic construct
  REGION 1..9: note = CDR H3 SEQUENCE
MPNWGSLDA

SEQ ID NO: 14 - protein, 9 aa, synthetic construct
  REGION 1..9: note = CDR H3 SEQUENCE
MPNWGSLDN

SEQ ID NO: 15 - protein, 9 aa, synthetic construct
  REGION 1..9: note = CDR H3 SEQUENCE
MPNWGALDS

SEQ ID NO: 16 - protein, 9 aa, synthetic construct
  REGION 1..9: note = CDR H3 SEQUENCE
MPNWGSLDN

SEQ ID NO: 17 - protein, 9 aa, synthetic construct
  REGION 1..9: note = CDR H3 SEQUENCE
MPNWGSLDY

SEQ ID NO: 18 - protein, 9 aa, synthetic construct
  REGION 1..9: note = CDR H3 SEQUENCE
MPNWGSFDH

SEQ ID NO: 19 - protein, 9 aa, synthetic construct
  REGION 1..9: note = CDR H3 SEQUENCE
MPNWGSLDS

SEQ ID NO: 20 - protein, 9 aa, synthetic construct
  REGION 1..9: note = CDR H3 SEQUENCE
MPNWGSLDG

SEQ ID NO: 21 - protein, 9 aa, synthetic construct
  REGION 1..9: note = CDR H3 SEQUENCE
VPNWGSLDG

SEQ ID NO: 22 - protein, 28 aa, synthetic construct
  REGION 1..28: note = CDR H3 SEQUENCE
CARFPNWGSLDHWGQGTLVTVSSASIKG

SEQ ID NO: 23 - protein, 28 aa, synthetic construct
  REGION 1..28: note = CDR H3 SEQUENCE
CARMPNWGSLDHWGQGTLVTVSSASTKG

SEQ ID NO: 24 - protein, 28 aa, synthetic construct
  REGION 1..28: note = CDR H3 SEQUENCE
CARMPNWGSFDYWGQGTLVTVSSASIKG

SEQ ID NO: 25 - protein, 12 aa, synthetic construct
  REGION 1..12: note = CDR H3 SEQUENCE
VRMPNWGSLDHW

SEQ ID NO: 26 - protein, 27 aa, synthetic construct
  REGION 1..27: note = CDR H3 SEQUENCE
VRMPNWGSLDHWGQGTLVTVSSADIKG

SEQ ID NO: 27 - protein, 27 aa, synthetic construct
  REGION 1..27: note = CDR H3 SEQUENCE
ARMPNWGSLDHWGQGTLVTVSSASIKG

SEQ ID NO: 28 - protein, 25 aa, synthetic construct
  REGION 1..25: note = CDR H3 SEQUENCE
FPNWGSFDYWGQGTLVTVSSASIKG

SEQ ID NO: 29 - protein, 9 aa, synthetic construct
  REGION 1..9: note = CDR H3 SEQUENCE
VPNWGSLDA

SEQ ID NO: 30 - protein, 9 aa, synthetic construct
  REGION 1..9: note = CDR H3 SEQUENCE
FPNWGSFDY

SEQ ID NO: 31 - protein, 12 aa, synthetic construct
  REGION 1..12: note = CDRL1
RASQSISSSYLA

SEQ ID NO: 32 - protein, 7 aa, synthetic construct
  REGION 1..7: note = CDRL2
GASSRAT

SEQ ID NO: 33 - skipped

SEQ ID NO: 34 - protein, 5 aa, synthetic construct
  REGION 1..5: note = CDRL3 SEQUENCE
QRYAT

SEQ ID NO: 35 - protein, 5 aa, synthetic construct
  REGION 1..5: note = CDRL3 SEQUENCE
QRYST

SEQ ID NO: 36 - protein, 5 aa, synthetic construct
  REGION 1..5: note = CDRL3 SEQUENCE
QMYST

SEQ ID NO: 37 - protein, 5 aa, synthetic construct
  REGION 1..5: note = CDRL3 SEQUENCE
QQVGT

SEQ ID NO: 38 - protein, 5 aa, synthetic construct
  REGION 1..5: note = CDRL3 SEQUENCE
QQVST

SEQ ID NO: 39 - protein, 5 aa, synthetic construct
  REGION 1..5: note = CDRL3 SEQUENCE
QQYST

SEQ ID NO: 40 - protein, 5 aa, synthetic construct
  REGION 1..5: note = CDRL3 SEQUENCE
QSYST

SEQ ID NO: 41 - protein, 5 aa, synthetic construct
  REGION 1..5: note = CDRL3 SEQUENCE
QQYAT

SEQ ID NO: 42 - protein, 5 aa, synthetic construct
  REGION 1..5: note = CDRL3 SEQUENCE
QQYSS

SEQ ID NO: 43 - protein, 5 aa, synthetic construct
  REGION 1..5: note = CDRL3 SEQUENCE
QTYST

SEQ ID NO: 44 - protein, 5 aa, synthetic construct
  REGION 1..5: note = CDRL3 SEQUENCE
QQYGS

SEQ ID NO: 45 - protein, 5 aa, synthetic construct
  REGION 1..5: note = CDRL3 SEQUENCE
QQYAS

SEQ ID NO: 46 - protein, 5 aa, synthetic construct
  REGION 1..5: note = CDRL3 SEQUENCE
QQYEA

SEQ ID NO: 47 - protein, 5 aa, synthetic construct
  REGION 1..5: note = CDRL3 SEQUENCE
QQYET

SEQ ID NO: 48 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 61 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC